SEQUENCE LISTING (9 sequences)

SEQ ID NO: 1 - protein, 451 aa, Homo sapiens
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKTRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1353 bp, Homo sapiens
  source 1..1353: mol_type = other DNA
caggtcaccctccgcgagagcggccctgctctcgtcaagcccacgcagaccctcacgctgacctgcaccttcagcggcttcagcctcagcaccagcggcatgagcgtcggctggatccgccagcctcctggcaaggccctcgagtggctcgccgacatctggtgggacgacaagaaggactacaaccccagcctcaagagccgcctcaccatcagcaaggacaccagcaagaaccaggtcgtcctgaaggtcaccaacatggaccccgccgacaccgccacctactactgcgcccgcagcatgatcaccaactggtacttcgacgtctggggcgctggcacgaccgtcaccgtcagcagcgcctccacgaagggccccagcgtctttccgctcgctcccagcagcaagagcacctccggcggcacggctgccctcggctgcctggtcaaggactacttccccgagcctgtcacggtcagctggaactctggcgccctgaccagcggcgtccacacgttccccgccgtcctccagagcagcggcctctactccctcagcagcgtcgtcacggtccccagcagctccctcggcacccagacctacatctgcaacgtcaaccacaagcctagcaacaccaaggtcgacacccgcgtcgagcccaagagctgcgacaagacccacacgtgccctccgtgccctgctcctgagctgcttggcggcccttcggtctttctgttccctccgaagcctaaggacaccctcatgatctcgcgcacgcccgaggtcacgtgcgtcgtcgtcgacgtcagccacgaggaccctgaggtcaagtttaactggtatgtcgacggcgtcgaggtccacaacgctaagacgaagccccgcgaggagcagtacaacagcacctaccgcgtcgtcagcgtcctcaccgtcctgcaccaggactggctcaacggcaaggagtacaagtgcaaggtcagcaacaaggccctgcctgctcctatcgagaagaccatctccaaggccaagggccagcctcgcgagccccaggtctacaccctgcctccgagccgagaggagatgacgaagaaccaggtgagcctgacctgcctcgtcaagggcttctaccccagcgacattgccgtcgagtgggagagcaacggccagcctgagaacaactacaagaccacgcctcctgtcctcgactccgacggctcgttcttcctgtacagcaagctcaccgtcgacaagtcccgctggcagcagggcaacgtctttagctgcagcgtcatgcacgaggccctccacaaccactacacccagaagtccctctcgctcagccccggcaagtaa

SEQ ID NO: 3 - protein, 450 aa, Homo sapiens
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDTRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 642 bp, Homo sapiens
  source 1..642: mol_type = other DNA
gacatccagatgacgcagagccccagcacgctcagcgccagcgtcggcgaccgcgtcaccatcacgtgcaagtgccagctctccgtcggctacatgcactggtatcagcagaagcccggcaaggcccctaagctcctcatctacgacaccagcaagctcgccagcggcgtccccagccgattctccggctctggctccggcaccgagttcaccctcaccatcagctcgctgcagcccgacgacttcgccacctactactgcttccagggctcgggctaccccttcaccttcggcggcggcacgaagctcgagatcaagcgcaccgtcgccgctcctagcgtctttatcttcccgcctagcgacgagcagctcaagagcggcaccgcctccgtcgtctgcctgctcaacaacttctacccgcgcgaggccaaggtccagtggaaggtcgacaacgccctccagagcggcaactcccaggagagcgtcaccgagcaggactccaaggacagcacctacagcctcagcagcaccctcacgctctccaaggccgactacgagaagcacaaggtctacgcctgcgaggtcacccaccagggcctgagcagccccgtcaccaagagcttcaaccgcggcgagtgctaa

SEQ ID NO: 5 - protein, 213 aa, Homo sapiens
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 1356 bp, Homo sapiens
  source 1..1356: mol_type = other DNA
gaggtccagctccaggagagcggcggcggcctcatgcagcccggcggcagcatgaagctctcctgcgtcgccagcggcttcaccttcagcaactactggatgaactgggtccgccagagccccgagaagggcctcgagtgggtcgccgagatccgcctcaagagcaacaactacgccacccactacgccgagagcgtcaagggccgcttcaccatcagccgcgacgacagcaagaactccgtctacctccagatgaacaccctccgcgccgaggacaccggcatctactactgcacgcgcggtaacggcaactaccgcgccatggactactggggccagggcaccagcgtcacggtctccagcgccagcaccaagggcccaagcgtctttcccctcgcccccagcagcaagagcaccagcggcggcaccgccgccctcggctgcctcgtcaaggactacttccccgagcccgtcactgtcagctggaacagcggcgctctcaccagcggcgtccacaccttccccgccgtcctccagagcagcggcctctacagcctcagcagcgtcgtcaccgtccccagcagcagcctcggcacccagacctacatctgcaacgtcaaccacaagcccagcaacaccaaggtcgacaagaccgtcgagcccaagagctgcgacaagacccacacctgccccccctgccccgcccccgagctgctcggcggcccctccgtctttctcttcccccccaagcccaaggacaccctcatgatcagccgcacccccgaggtcacctgcgtcgtcgtcgatgtcagccacgaggaccccgaggtcaagttcaactggtacgtcgacggcgtcgaggtccacaacgccaagaccaagccccgcgaggagcagtacaacagcacctaccgcgtcgtcagcgtcctgaccgtcctccaccaggactggctcaacggcaaggagtacaagtgcaaggtctccaacaaggccctccccgcccccatcgaaaagaccatcagcaaggccaagggccagccccgcgagccccaggtctacaccctcccccccagccgcgaggagatgaccaagaaccaggtctccctcacctgcctggtcaagggcttctaccccagcgacatcgccgtcgagtgggagagcaacggccagcccgagaacaactacaagaccaccccccccgtcctcgacagcgacggcagcttcttcctctacagcaagctcaccgtcgacaagagccgctggcagcagggcaacgtctttagctgcagcgtcatgcacgaggccctccacaaccactacacccagaagagcctcagcctcagccccggcaagtaa

SEQ ID NO: 7 - protein, 451 aa, Homo sapiens
EVQLQESGGGLMQPGGSMKLSCVASGFTFSNYWMNWVRQSPEKGLEWVAEIRLKSNNYATHYAESVKGRFTISRDDSKNSVYLQMNTLRAEDTGIYYCTRGNGNYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 645 bp, Homo sapiens
  source 1..645: mol_type = other DNA
gacatccagatgacccagagccccgccagcctcagcgtctctgtcggcgagaccgtctccatcacctgccgcgccagcgagaacatctacagcagcctcgcctggtatcagcagaagcagggcaagagcccccagctcctcgtctacagcgccaccatcctcgccgacggcgtccccagccgcttcagcggcagcggcagcggcacccagtacagcctcaagatcaacagcctccagagcgaggacttcggcacctactactgccagcacttctggggcaccccctacaccttcggcggcggcaccaagctggagatcacccgcaccgtcgcggcgccaagcgtctttatcttcccccccagcgacgagcagctcaagagcggcaccgccagcgtcgtctgcctcctcaacaacttctacccccgcgaggccaaggtccagtggaaggtcgacaacgccctccagagcggcaacagccaggagagcgtcaccgagcaggacagcaaggactccacctacagcctcagcagcaccctcaccctctccaaggccgactacgagaagcacaaggtctacgcctgcgaggtcacccaccagggcctcagctcccccgtcaccaagagcttcaaccgcggcgagtgctaa

SEQ ID NO: 9 - protein, 214 aa, Homo sapiens
DIQMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0285948 A1
(43) Pub. Date: Sep. 24, 2026

(54) ENGINEERED ANTIBODIES

(71) Applicant: DANISCO US INC, Palo Alto, CA (US)

(72) Inventors: Jimmy CHAN, SAN MATEO, CA (US); David A. ESTELL, SAN MATEO, CA (US); Jeffrey Wayne MUNOS, SAN FRANCISCO, CA (US); Michael C. MILLER, SAN FRANCISCO, CA (US); Igor NIKOLAEV, NOORDWIJK (NL)

(21) Appl. No.: 19/629,188

(22) Filed: Mar. 26, 2026

Related U.S. Application Data
(63) Continuation of application No. 17/599,135, filed on Mar. 30, 2020, now abandoned.
(60) Provisional application No. 62/825,448, filed on Mar. 28, 2019.

(51) Int. Cl. C07K 16/00; C07K 16/10; C12N 9/42

(52) U.S. Cl. CPC C07K 16/00; C07K 16/10; C12N 9/2437; C12Y 302/01091; C07K 2317/24; C07K 2317/53; C07K 2317/94; C07K 2319/02; C07K 2319/35

(57) ABSTRACT

Disclosed herein, inter alia, are cleavage-resistant antibodies and antibody-producing host cells as well as methods for making, using, and improving secretion of the same.

### CROSS-REFERENCE TO RELATED APPLICATIONS

This application if a Continuation of U.S. application Ser. No. 17/599,135, which is a U.S. National Stage Application under 35. U.S.C. § 371 of International Patent Application No. PCT/US2020/025638, filed Mar. 30, 2020, which claims priority to U.S. Provisional Patent Application No. 62/825,448, filed Mar. 28, 2019, the disclosure of which is incorporated by reference herein in its entirety.

### INCORPORATION BY REFERENCE OF THE SEQUENCE LISTING

The present application is being filed with a Sequence Listing in electronic format. The Sequence Listing is provided as a file entitled 20260325_NB41596USPCN_SeqLst.xml, created on Mar. 25, 2026, which is 14,440 bytes in size. The information in the electronic format of the Sequence Listing is incorporated by reference in its entirety.

### FIELD OF THE INVENTION

Provided herein, inter alia, are antibodies with modified hinge regions, wherein the antibody is more resistant to cleavage and/or has increased stability relative to an identical antibody having an unmodified hinge region.

### BACKGROUND

Antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events. The constant region of the heavy chain may be further divided into four smaller domains called: CH1, the hinge region, CH2 and CH3. A portion of the constant region, the Fc region, is involved in a number of important cellular functions. Generally, the Fc region is defined as only comprising CH2 and CH3 and may also encompass a portion of the hinge region.

Antibodies of the IgG isotype are exceptionally flexible molecules. Indeed, the biological function of IgGs requires very specific and controlled modes of deformation. The structure primarily responsible for the internal flexibility of IgG molecules is located between the first (CH1) and second (CH2) domains of the constant region, and is termed the hinge region. The hinge can be divided into three peptide regions; upper, middle and lower hinge respectively Brekke et al., 1995, Immunol Today 16:85-90. Biochemical and structural studies point to the hinge region of antibodies as a key structural element that control flexibility and modulates effector functions. The crystal structure of IgG1 b12 (Saphire et al., 2001, Science 293:1155-1159 and Saphire et al., 2002, Journal of Molecular Biology, 319(1): 9-18) revealed extreme asymmetry, indicative of the extraordinary interdomain flexibility within the antibody. The structure of b12 revealed another characteristic of the hinge, which is its frailty; the structure of b12 shows extensive damage in the hinge region, consistent with prior knowledge of antibody hinge properties.

Therefore, it is not surprising that when recombinant antibodies are expressed in host cells, significant proteolytic cleavage (i.e. clipping) events in the antibody hinge region are often observed. Such cleavage makes the antibody production process less efficient and results in decreased titers of functional immunoglobulin. What is needed, therefore, are improved compositions and methods for decreasing the amount of proteolytic clipping in the hinge regions of host cell-expressed antibodies during the production and purification processes. The subject matter disclosed herein addresses these needs and provides additional benefits as well.

### SUMMARY

Provided herein, inter alia, are non-naturally occurring engineered monoclonal antibodies with altered amino acid sequences in the antibody hinge region with decreased or no protease-mediated cleavage (i.e. clipping) during production and purification as well as methods for producing the same. The disclosed methods, engineered antibodies, and recombinant host cells result in increased antibody production and/or purification when compared to antibodies that do not contain the disclosed altered hinge region amino acid sequences and/or that are not used in accordance with the methods disclosed herein.

Accordingly, in some aspects, provided herein is a monoclonal IgG1 antibody heavy chain polypeptide comprising a hinge region that comprises one or more amino acid modification(s) that reduces proteolysis of the polypeptide. In some embodiments, the polypeptide further comprises an IgG1 antibody light chain polypeptide. In some embodiments of any of the embodiments disclosed herein, the modification(s) comprise a modification at one or more of amino acid positions 216, 217, 222, 226, and/or 234, wherein the amino acid positions are numbered according to the numbering in SEQ ID NO:1. In some embodiments, the modifications comprise one or more of 216T or V; 217T or S; 222C, D, or E; 226N or P; and/or 234R. In some embodiments of any of the embodiments disclosed herein, the modification further comprises a modification at position 227. In some embodiments, the modification comprises 227P. In some embodiments of any of the embodiments disclosed herein, the modifications comprise a modification at position 216 and one or more modifications at amino acid positions 222, 226, 227, and/or 234. In some embodiments, the modifications comprise 216T and one or more of 222C, D, or E; 226N or P; 227P; and/or 234R. In some embodiments of any of the embodiments disclosed herein, the modifications comprise a modification at position 217 and one or more modifications at amino acid positions 222, 226, 227, and/or 234. In some embodiments, the modifications comprise 217T and one or more of 222C, D, or E; 226N or P; 227P; and/or 234R. In some embodiments of any of the embodiments disclosed herein, the modification is a combinatorial modification selected from the group consisting of: (a) R217S-T226N; (b) R217S-S222C; (c) T216V-R217S-T226N; (d) S222C-H227P; (e) R217S-S222E-H227P; (f) T216V-R217S-S222C-T226P; (g) T226P-H227P; (h) R217S-S222C-T226P; (i) T216V-S222D-T226P; (j) T216V-S222C-T226P-H227P; (k) T216V-S222E-T226N; (1) T216V-S222C-H227P; (m) R217S-S222C-T226N; (n) S222C-T226P-H227P; (o) T216V-S222C-T226N-H227P; (p) R217S-S222D-T226P-H227P; (q) T216V-H227P; (r) S222E-T226P-H227P; (s) R217S-S222D-T226N-H227P; (t) R217S-S222E-T226P-H227P; (u) T216V-S222C; (v) R217S-S222D; (w) S222E-T226P; (x) R217S-S222C-T226P-H227P; (y) T216V-T226P-H227P; (z) T216V-S222D-H227P; (aa) T226N-H227P; (bb) S222D-T226N-H227P; (cc) R217S-S222E-T226N; (dd) R217S-T226N-H227P; (ee) R217S-S222E-T226N-H227P; (ff) T216V-R217S-H227P; (gg) T216V-S222E-T226P-H227P; (hh) T216V-S222C-T226N; (ii) R217S-T226P-H227P; (jj) T216V-T226P; (kk) S222E-T226N-H227P; (11) S222E-H227P; (mm) R217S-S222E-T226P; (nn) R217S-T226P; (00) T216V-S222D; (pp) R217S-S222C-H227P; (qq) T216V-S222E-T226N-H227P; (rr) S222C-T226N-H227P; (ss) T216V-R217S-S222C-T226N-H227P; (tt) R217S-S222D-T226P; and (uu) S222D-T226N (vv). In some embodiments of any of the embodiments disclosed herein, said polypeptide exhibits at least about 50% less proteolysis compared to a monoclonal IgG1 antibody heavy chain polypeptide that does not comprise said one or more amino acid modifications. In some embodiments of any of the embodiments disclosed herein, said polypeptide exhibits no detectable proteolysis. In some embodiments of any of the embodiments disclosed herein, the polypeptide further comprises a polypeptide encoding a signal sequence. In some embodiments of any of the embodiments disclosed herein, the polypeptide further comprised a polypeptide encoding a carrier protein. In some embodiments of any of the embodiments disclosed herein, the polypeptide encoding a carrier protein is adjacent to the polypeptide encoding a signal sequence. In some embodiments of any of the embodiments disclosed herein, the carrier protein comprises CBH1 or a fragment thereof. In some embodiments of any of the embodiments disclosed herein, the antibody is an anti-Respiratory Syncytial Virus (RSV) antibody, an anti-ebola virus antibody, an anti-aggregated β-amyloid (AB) antibody, an anti-human immunodeficiency virus (HIV) antibody, an anti-herpes simplex virus (HSV) antibody, an anti-sperm antibody (such as an anti-human contraceptive antigen (HCA) antibody), or an anti-HER2/neu antibody. In some embodiments of any of the embodiments disclosed herein, the polypeptide exhibits increased stability compared to a monoclonal IgG1 antibody heavy chain polypeptide that does not comprise said one or more amino acid modifications.

In other aspects, provided herein is a nucleic acid encoding any of the polypeptides disclosed herein. In still further aspects, provided herein is a vector encoding any of the nucleic acids disclosed herein. In some embodiments, the vector further comprises a nucleic acid sequence encoding a promoter.

In another aspect, provided herein is a host cell comprising any of the polypeptides disclosed herein, any of the nucleic acids disclosed herein, or any of the vectors disclosed herein. In some embodiments, the host cell is selected from the group consisting of a mammalian host cell, a bacterial host cell, and a fungal host cell. In some embodiments, the mammalian cell is a Chinese Hamster Ovary (CHO) cell. In some embodiments, the bacterial cell is an E. coli cell. In some embodiments, the fungal cell is a yeast cell or a filamentous fungal cell. In some embodiments, the yeast cell is a Saccharomyces sp. In some embodiments of any of the embodiments disclosed herein, the fungal cell is selected from the group consisting of a Trichoderma sp., a Penicillium sp., a Humicola sp., a Chrysosporium sp., a Gliocladium sp., an Aspergillus sp., a Fusarium sp., a Mucor sp., a Neurospora sp., a Hypocrea sp.; Myceliophthora sp., and an Emericella sp. In some embodiments, the fungal cell is selected from the group consisting of Trichoderma reesei, Trichoderma viride, Trichoderma koningii, Trichoderma harzianum, Humicola insolens, Humicola grisea, Chrysosporium lucknowense, Aspergillus oryzae, Aspergillus niger, Aspergillus nidulans, Aspergillus kawachi, Aspergillus aculeatus, Aspergillus japonicus, Aspergillus sojae, Myceliophthora thermophila, and Aspergillus awamori.

In further aspects, provided herein is a method for producing any of the polypeptides disclosed herein comprising: culturing any of the host cells disclosed herein under suitable conditions for the production of the polypeptide. In some embodiments, the method further comprises isolating the polypeptide. In some embodiments of any of the embodiments disclosed herein, said polypeptide exhibits at least about 50% less proteolysis compared to a monoclonal IgG1 antibody heavy chain polypeptide that does not comprise said one or more amino acid modifications. In some embodiments of any of the embodiments disclosed herein, said polypeptide exhibits no detectable proteolysis.

In some aspects, provided herein is a method for modifying a monoclonal IgG1 antibody heavy chain polypeptide to increase its resistance to proteolysis comprising modifying one or more amino acid residues in a hinge region of the polypeptide. In some embodiments, the modification(s) comprise a modification at one or more of amino acid positions 216, 217, 222, 226, and/or 233, wherein the amino acid positions are numbered according to the numbering in SEQ ID NO:1. In some embodiments, the modifications comprise one or more of 216T or V; 217S; 222C, D, or E; 226N or P; and/or 234R. In some embodiments, the modification further comprises 227P. In some embodiments of any of the embodiments disclosed herein, the modification is a combinatorial modification selected from the group consisting of: (a) R217S-T226N; (b) R217S-S222C; (c) T216V-R217S-T226N; (d) S222C-H227P; (e) R217S-S222E-H227P; (f) T216V-R217S-S222C-T226P; (g) T226P-H227P; (h) R217S-S222C-T226P; (i) T216V-S222D-T226P; (j) T216V-S222C-T226P-H227P; (k) T216V-S222E-T226N; (l) T216V-S222C-H227P; (m) R217S-S222C-T226N; (n) S222C-T226P-H227P; (o) T216V-S222C-T226N-H227P; (p) R217S-S222D-T226P-H227P; (q) T216V-H227P; (r) S222E-T226P-H227P; (s) R217S-S222D-T226N-H227P; (t) R217S-S222E-T226P-H227P; (u) T216V-S222C; (v) R217S-S222D; (w) S222E-T226P; (x) R217S-S222C-T226P-H227P; (y) T216V-T226P-H227P; (z) T216V-S222D-H227P; (aa) T226N-H227P; (bb) S222D-T226N-H227P; (cc) R217S-S222E-T226N; (dd) R217S-T226N-H227P; (ee) R217S-S222E-T226N-H227P; (ff) T216V-R217S-H227P; (gg) T216V-S222E-T226P-H227P; (hh) T216V-S222C-T226N; (ii) R217S-T226P-H227P; (jj) T216V-T226P; (kk) S222E-T226N-H227P; (ll) S222E-H227P; (mm) R217S-S222E-T226P; (nn) R217S-T226P; (oo) T216V-S222D; (pp) R217S-S222C-H227P; (qq) T216V-S222E-T226N-H227P; (rr) S222C-T226N-H227P; (ss) T216V-R217S-S222C-T226N-H227P; (tt) R217S-S222D-T226P; and (uu) S222D-T226N (vv). In some embodiments of any of the embodiments disclosed herein, said polypeptide exhibits at least about 50% less proteolysis compared to a monoclonal IgG1 antibody heavy chain polypeptide that does not comprise said one or more amino acid modifications. In some embodiments of any of the embodiments disclosed herein, said polypeptide exhibits no detectable proteolysis. In some embodiments of any of the embodiments disclosed herein, said polypeptide exhibits increased stability compared to a monoclonal IgG1 antibody heavy chain polypeptide that does not comprise said one or more amino acid modifications.

In further aspects, provided herein is a monoclonal IgG1 antibody heavy chain polypeptide produced by any of the methods disclosed herein.

In yet further aspects, provided herein is a kit comprising a) written instructions for producing any of the polypeptides disclosed herein; and b) one or more of 1) any of the nucleic acids disclosed herein; 2) any of the vectors disclosed herein; and/or 3) any of the host cells disclosed herein.

In another aspect, provided herein is a syringe, cannula, or catheter comprising any of the polypeptides disclosed herein.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts a P Entry clone used for Synagis HC heavy chain SEL library construction.

FIG. 2 depicts the expression vector pTTTpyr2-IScel-Synagis HC_Geneart_SEL heavy chain.

FIG. 3 depicts a P Entry clone used for c2G4_HC3 SEL library construction.

FIG. 4 depicts the expression vector to produce c2G4_HC3 heavy chain.

FIG. 5 depicts the expression cassette of c2G4_LC2 light chain.

FIG. 6 depicts a graph showing the reduction in hinge clipping for engineered C2G4 variants versus wildtype C2G4 control samples. The x-axis displays the final sum of bands, which is used to confirm that the calculated extent of clipping is based on significant bands and not noise. The drawn solid line is the average delta clipping for the WT samples, and the dashed lines are plus and minus 1 standard deviation of the average WT.

FIG. 7 depicts a graph highlighting the reduction in hinge clipping for the engineered variants versus wildtype control samples. The x-axis displays the final sum of bands, which is used to confirm that the calculated extent of clipping is based on significant bands and not noise. The square data points are for pooled WT-hinge samples. Each data point represents biological replicates that were pooled after harvest, but were then purified and assayed independently. The “+” data point is for HC:W105F, which is known variant that has a stronger binding interaction with antigen. This variant has a wildtype hinge sequence. The triangle data point is a biological WT-hinge replicate that was not pooled before it was purified and assayed. The circle data points represent the engineered variants listed in Table 4.

FIG. 8 depicts a graph showing the reduction in hinge clipping for engineered variants versus wildtype hinge control samples. The x-axis displays the final sum of bands, which is used to confirm that the calculated extent of clipping is based on significant bands and not noise.

FIG. 9 depicts Sequence alignment for the hinge region of antiRSV and C2G4.

### DETAILED DESCRIPTION

The invention disclosed herein is based, in part, on the inventors' observations that undesirable antibody cleavage is eliminated or decreased when a wildtype (i.e., a naturally-occurring) antibody hinge region domain is engineered to include one or more alternative substituted amino acids.

Accordingly, provided herein are DNA constructs, vectors, antibodies, host cells expressing DNA constructs and/or cleavage-resistant antibodies, as well as methods for enhancing the secretion and/or preventing or decreasing the unwanted cleavage of an antibody. More specifically, and in some non-limiting aspects, engineered antibody hinge sequences have been included in an antibody to prevent or decrease cleavage (i.e. clipping) of antibodies during host cell production. The antibodies disclosed herein exhibit better secretion, stability, and/or purification compared to antibodies that do not include the engineered hinge sequences disclosed herein. As such, the instant disclosure provides alternative and improved methods for antibody production, particularly therapeutic protein production, which result in high levels of purified antibodies with limited risk of contamination by unwanted cleavage products.

### I. Definitions

The term “polypeptide” or “protein” is meant to refer to any polymer containing any of the 20 natural amino acids regardless of its size. Although the term “protein” is often used in reference to relatively large proteins, and “peptide” is often used in reference to small polypeptides, use of these terms in the field often overlaps. The term “polypeptide” thus refers generally to proteins, polypeptides, and peptides unless otherwise noted. The conventional one-letter or three-letter code for amino acid residues is used herein.

The term “nucleic acid” or “polynucleotide” encompasses DNA, RNA, single stranded or double stranded and chemical modifications thereof. The terms “nucleic acid” and “polynucleotide” can be used interchangeably herein. Because the genetic code is degenerate, more than one codon can be used to encode a particular amino acid, and the present subject matter encompasses polynucleotides, which encode a particular amino acid sequence.

As used herein, the terms “antibody” and “antibodies” refer to monoclonal antibodies, multispecific antibodies, human antibodies, humanized antibodies, camelised antibodies, chimeric antibodies, single-chain Fvs (scFv), disulfide-linked Fvs (sdFv), Fab fragments, F(ab′) fragments, and anti-idiotypic (anti-Id) antibodies (including, e.g., anti-Id antibodies to the engineered antibodies disclosed herein), and epitope-binding fragments of any of the above. In particular, antibodies include immunoglobulin molecules and immunologically active fragments of immunoglobulin molecules, i.e., molecules that contain an antigen binding site, these fragments may or may not be fused to another immunoglobulin domain including but not limited to, an Fc region or fragment thereof. As outlined herein, the terms “antibody” and “antibodies” specifically include the hinge region variants described herein, full length antibodies and hinge variant-fusions comprising a modified hinge as described herein fused to an immunologically active fragment of an immunoglobulin or to other proteins. Such Fe variant-fusions include but are not limited to, scFv-Fc fusions, variable region (e.g., VL and VH)-Fc fusions, scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the “hinge region” is generally defined as stretching from 212-238 (EU numbering) or 222-251 (Kabat numbering) of human IgG1. The hinge may be further divided into three distinct regions, the upper, middle and lower hinge. In some embodiments, the hinge region is defined as stretching from amino acid 216-238 of the sequence shown in SEQ ID NO:1. Hinge regions of other IgG isotypes may be aligned with the IgG 1 sequence or the sequence shown in SEQ ID NO: 1 using any number of publicly available sequence alignment programs.

The terms “proteolysis,” “proteolytic degradation,” “cleavage,” and “clipping” as used interchangeably herein in the context of antibody production refer to the unwanted breakdown of antibody polypeptide components (such as an antibody heavy chain polypeptide) into smaller polypeptides that are generally considered undesirable byproducts of antibody expression and production in recombinant host cells (such as filamentous fungal host cell). In some embodiments, the breakdown can occur by cleavage of peptide bonds located in the antibody heavy chain hinge region due to enzymatic or chemical mechanisms. In alternative embodiments, the breakdown may occur by cleavage of crosslinks between homologous or heterologous proteins. In some embodiments, proteolysis occurs during antibody expression in a host cell (such as a eukaryotic, for example, a mammalian or fungal host cell). In other embodiments, proteolysis occurs during or subsequent to isolation and/or purification of the antibody.

“Stability” and “stable” refer to the resistance of engineered antibodies in a formulation to aggregation, degradation or fragmentation under given manufacture, preparation, transportation and storage conditions. An engineered antibody with improved stability will retain biological activity under given manufacture, preparation, transportation and storage conditions. The stability of an engineered antibody can be assessed by degrees of aggregation, degradation or fragmentation, as measured by High Performance Size Exclusion Chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques. The stability of an engineered antibody may be compared to a comparable molecule under identical conditions. The overall stability of an engineered antibody can also be assessed by various immunological assays including, for example, ELISA and radioimmunoassay using isolated antigen molecules or cells expressing the same.

“The terms “wild-type,” “wildtype,” “parental,” or “reference,” with respect to a polypeptide, refer to a naturally-occurring polypeptide that does not include a man-made substitution, insertion, or deletion at one or more amino acid positions. Similarly, the term “wild-type,” “wildtype,” “parental,” or “reference,” with respect to a polynucleotide, refers to a naturally-occurring polynucleotide that does not include a man-made nucleoside change. However, a polynucleotide encoding a wild-type, parental, or reference polypeptide is not limited to a naturally-occurring polynucleotide, but rather encompasses any polynucleotide encoding the wild-type, parental, or reference polypeptide.

As used herein, the term “non-naturally occurring” refers to anything that is not found in nature (e.g., recombinant nucleic acids and protein sequences produced in the laboratory), such as the modification of a wild-type nucleic acid and/or amino acid sequence. In some embodiments, a non-naturally occurring polypeptide contains an amino acid substitution (i.e. a mutation) that is not found in a corresponding wild-type or naturally-occurring amino acid sequence.

As used herein, a “derivative” or “variant” of a polypeptide means a polypeptide, which is derived from a precursor polypeptide (e.g., the native polypeptide) by addition of one or more amino acids to either or both the C- and N-terminal end, substitution of one or more amino acids at one or a number of different sites in the amino acid sequence, deletion of one or more amino acids at either or both ends of the polypeptide or at one or more sites in the amino acid sequence, or insertion of one or more amino acids at one or more sites in the amino acid sequence.

As used herein, a “variant polynucleotide” encodes a variant polypeptide, has a specified degree of homology/identity with a parent polynucleotide, or hybridized under stringent conditions to a parent polynucleotide or the complement thereof. Suitably, a variant polynucleotide has at least 80%, at least 85%, at least 90%, at least 91%, at least 92%, at least 93%, at least 94%, at least 95%, at least 96%, at least 97%, at least 98%, or even at least 99% nucleotide sequence identity to a parent polynucleotide or to a complement of the parent polynucleotide. Methods for determining percent identity are known in the art.

The term “derived from” encompasses the terms “originated from,” “obtained from,” “obtainable from,” “isolated from,” and “created from,” and generally indicates that one specified material finds its origin in another specified material or has features that can be described with reference to another specified material.

“Control sequence” is defined herein to include all components, which are necessary or advantageous for the expression of a polynucleotide or polypeptide of interest. Each control sequence can be native or foreign to the nucleic acid sequence encoding a polypeptide. Such control sequences include, but are not limited to, a leader sequence, polyadenylation sequence, propeptide sequence, promoter, signal peptide sequence, and transcription terminator. At a minimum, the control sequences include a promoter, and transcriptional and translational stop signals. The control sequences can be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed in a functional relationship (i.e., at a position relative to) with a polynucleotide or polypeptide of interest, such as the coding sequence in the DNA sequence, such that the control sequence directs or regulates the expression of a polynucleotide and/or polypeptide.

The term “DNA construct” means a DNA sequence which is operably linked to a suitable control sequence capable of effecting expression of a protein in a suitable host. Such control sequences can include a promoter to effect transcription, an optional operator sequence to control transcription, a sequence encoding suitable ribosome binding sites on the mRNA, enhancers and sequences which control termination of transcription and translation.

The term “fusion DNA construct” or “fusion nucleic acid” refers to a nucleic acid which comprises from 5′ to 3′ a number of polynucleotide sequences (e.g. and without limitation, a DNA molecule encoding a signal sequence, a DNA molecule encoding a carrier protein, a DNA molecule coding for a KEX2 site and a DNA molecule encoding a polypeptide of interest) operably linked together and which encode a fusion polypeptide.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a vector that has the ability to incorporate and express heterologous DNA fragment in a foreign cell. Many prokaryotic and eukaryotic expression vectors are commercially available.

“Promoter” or “promoter sequence” is a nucleic acid sequence that is recognized by a host cell for expression of a polynucleotide of interest, such as a coding region. Generally, the promoter sequence contains transcriptional control sequences, which mediate the expression of a polynucleotide of interest. The promoter can be any nucleic acid sequence which shows transcriptional activity in the host cell of choice, including mutant, truncated, and hybrid promoters, and can be obtained from genes encoding extracellular or intracellular polypeptides either homologous or heterologous to the host cell.

The term “signal sequence” refers to a sequence of amino acids at the amino terminus of a protein that directs the protein to the secretion system for secretion from a cell. The signal sequence is cleaved from the protein prior to secretion of the protein. In certain cases, a signal sequence can be referred to as a “signal peptide” or “leader peptide”. The definition of a signal sequence is a functional one. The mature form of the extracellular protein lacks the signal sequence which is cleaved off during the secretion process.

The term “carrier protein” as used herein refers to proteins that function to or facilitate the folding and secretion of polypeptides from a host cell. Exemplary carrier proteins are discussed in more detail below.

The term “recombinant,” when used in reference to a subject cell, nucleic acid, polypeptides/enzymes or vector, indicates that the subject has been modified from its native state. Thus, for example, recombinant cells express genes that are not found within the native (non-recombinant) form of the cell, or express native genes at different levels or under different conditions than found in nature. Recombinant nucleic acids can differ from a native sequence by one or more nucleotides and/or are operably linked to heterologous sequences, e.g., a heterologous promoter, signal sequences that allow secretion, etc., in an expression vector. Recombinant polypeptides/enzymes can differ from a native sequence by one or more amino acids and/or are fused with heterologous sequences. A vector comprising a nucleic acid encoding an antibody heavy chain is, for example, a recombinant vector.

As used herein, “microorganism” refers to a bacterium, a fungus, a virus, a protozoan, and other microbes or microscopic organisms.

“Host strain” or “host cell” means a suitable host for an expression vector or DNA construct comprising a polynucleotide encoding a polypeptide and particularly a recombinant polypeptide encompassed by the present disclosure. In specific embodiments, the host strains can be a filamentous fungal cell or a mammalian cell. The term “host cell” includes both cells and protoplasts.

The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, glucans, and other complex polysaccharides. The filamentous fungi disclosed herein are morphologically, physiologically, and genetically distinct from yeasts. Vegetative growth by filamentous fungi is by hyphal elongation and carbon catabolismis obligatory aerobic.

The term “culturing” refers to growing a population of microbial cells under suitable conditions in a liquid or solid medium.

The term “heterologous” with reference to a polynucleotide or polypeptide refers to a polynucleotide or polypeptide that does not naturally occur in a host cell. In some embodiments, the protein is a commercially important industrial protein and in some embodiments, the heterologous protein is a therapeutic protein. It is intended that the term encompass proteins that are encoded by naturally occurring genes, mutated genes, and/or synthetic genes.

The term “homologous” with reference to a polynucleotide or protein refers to a polynucleotide or protein that occurs naturally in the host cell.

The terms “recovered,” “isolated,” and “separated,” as used herein, refer to a protein (for example, a polypeptide of interest), cell, nucleic acid or amino acid that is removed from at least one component with which it is associated.

As used herein, the terms “transformed”, “stably transformed” and “transgenic” used in reference to a cell means the cell has a non-native (e.g., heterologous) nucleic acid sequence or additional copy of a native (e.g., homologous) nucleic acid sequence integrated into its genome or has an episomal plasmid that is maintained through multiple generations.

As used herein, the term “expression” refers to the process by which a polypeptide is produced based on the nucleic acid sequence of a gene. The process includes both transcription and translation.

The term “secreted protein” refers to a region of a polypeptide that is released from a cell during protein secretion.

The term “secretion” refers to the selective movement of a protein across a membrane in a host cell to the extracellular space and surrounding media.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

### A. Engineered Antibodies

Provided herein are non-naturally occurring antibodies, fragments thereof, or variants thereof with modified hinge regions having improved expression and/or cleavage properties. The modified hinge region may exhibit alterations in one or more of the characteristics of the hinge, including, but not limited to, stability, flexibility, length, conformation, charge, resistance to cleavage (such as proteolytic cleavage) and hydrophobicity relative to a wild type antibody hinge. The modified hinge regions disclosed herein may be generated by methods well known in the art, such as, for example introducing a modification into a wild type hinge. Modifications which may be utilized to generate a modified hinge region include, but are not limited to, amino acid insertions, deletions, substitutions, and rearrangements. Said modifications of the hinge and the modified hinge regions disclosed are referred to herein jointly as “hinge modifications” or simply “modified hinge(s).” The modified hinge regions disclosed herein may be incorporated into a molecule of choice including, but not limited to, antibodies and fragments thereof.

As demonstrated herein, molecules comprising a modified hinge may exhibit decreased or eliminated proteolysis during host cell production and/or purification when compared to a molecule having the same amino acid sequence except for the modified hinge such as, for example, a molecule having the same amino acid sequence except comprising a wild type hinge. As demonstrated herein, molecules comprising a modified hinge may have improved stability and/or storage (i.e., increased shelf life) and resistance to cleavage when compared to a molecule having the same amino acid sequence except for the modified hinge such as, for example, a molecule having the same amino acid sequence except comprising a wild type hinge.

In one embodiment, engineered antibodies will have at least a modified hinge (e.g., a hinge region comprising one or more amino acid insertions, deletions, substitutions, or rearrangements) wherein said engineered antibody has improved stability and/or resistance to cleavage relative to a comparable molecule.

The engineered antibodies disclosed herein encompass antibody variants comprising a hinge modification, said modification altering one or more characteristics of the hinge including, but not limited to, stability, resistance to cleavage (such as, proteolytic cleavage) flexibility, length, conformation, charge and hydrophobicity. The engineered antibodies disclosed herein also encompasses variants comprising a modified hinge, said modified hinge exhibiting one or more altered characteristics relative to a wild type hinge including, but not limited to, stability, flexibility, length, conformation, charge and hydrophobicity. The modified hinges disclosed may be generated by methods well know in the art, such as, for example, introducing a modification into a wild type hinge. Hinge modifications which may be utilized in generating a modified hinge include, but are not limited to, insertions, deletions, inversions and substitutions of one or more amino acid residues. It will be appreciated by one skilled in the art that combinations of insertions and/or deletions and/or substitutions may also be used to generate a modified hinge.

In certain embodiments, the engineered antibodies encompass hinge modifications which are the substitution of at least one amino acid residue in the hinge. In one embodiment, at least one, or at least two, or at least three, or at least four, or at least five, or at least ten, or at least 15 amino acid residues are substituted in the hinge. In one embodiment, the substitution is made in the upper hinge. In another embodiment, the substitution is made in the middle hinge. In another embodiment, the substitution is made in the lower hinge. In still another embodiment, substitutions are made in more than one position including, but not limited to, the upper hinge, the middle hinge and the lower hinge.

In still another embodiment, the engineered antibody comprises at least one substitution in the hinge region, wherein the substitution is located at an amino acid position selected from the group consisting of: 216, 217, 222, 226, 227, and/or 234, wherein the amino acid positions are numbered according to the numbering in SEQ ID NO:1 or at positions 213, 214, 221, 223, 224, and/or 231, wherein the numbering system is that of the EU index as set forth in Kabat, or at positions 223, 224, 232, 236, 237, and/or 244, wherein the numbering system is that of the Kabat index as set forth in Kabat. Any combination of the hinge modifications set forth in Table 1 and are specifically contemplated as embodiments.

TABLE 1
Combinations of substitutions

| | |
|---|---|
| R217S-T226N | T216V-S222D-H227P |
| R217S-S222C | T226N-H227P |
| T216V-R217S-T226N | S222D-T226N-H227P |
| S222C-H227P | R217S-S222E-T226N |
| R217S-S222E-H227P | R217S-T226N-H227P |
| T216V-R217S-S222C-T226P | R217S-S222E-T226N-H227P |
| T226P-H227P | T216V-R217S-H227P |
| R217S-S222C-T226P | T216V-S222E-T226P-H227P |
| T216V-S222D-T226P | T216V-S222C-T226N |
| T216V-S222C-T226P-H227P | R217S-T226P-H227P |
| T216V-S222E-T226N | T216V-T226P |
| T216V-S222C-H227P | S222E-T226N-H227P |
| R217S-S222C-T226N | S222E-H227P |
| S222C-T226P-H227P | R217S-S222E-T226P |
| T216V-S222C-T226N-H227P | R217S-T226P |
| R217S-S222D-T226P-H227P | T216V-S222D- |
| T216V-H227P | R217S-S222C-H227P |
| S222E-T226P-H227P | T216V-S222E-T226N-H227P |
| R217S-S222D-T226N-H227P | S222C-T226N-H227P |
| R217S-S222E-T226P-H227P | T216V-R217S-S222C-T226N-H227P |
| T216V-S222C | R217S-S222D-T226P |
| R217S-S222D | S222D-T226N |
| S222E-T226P | R217S-S222C-T226P-H227P |
| T216V-T226P-H227P | |

In one embodiment, the engineered antibodies disclosed herein comprise a modified hinge that has altered (e.g., increased or decreased) flexibility of the hinge, relative to a wild type hinge. A modified hinge having altered flexibility of the hinge may be generated by incorporating certain modifications into a wild type hinge. Hinge modifications which increase the flexibility of the hinge include but are not limited to, the substitution of one or more amino acids residues with one or more amino acid residues which increase the flexibility (e.g., Glycine), the substitution of a cysteine involved in the formation of a disulfide bond with an amino acid residue which can not form a disulfide bond (e.g. Serine, Alanine, Glycine), the insertion of one or more amino acid residues which allow for a high degree of local flexibility (e.g., Glycine) and the deletion of one or more amino acid residues which increase the rigidity of a polypeptide (e.g., Proline). Hinge modifications which decrease the flexibility of the hinge include but are not limited to the substitution of one or more amino acids residues with one or more amino acid residues which increase the rigidity of the polypeptide (e.g., Proline), the substitution of an amino acid residue which cannot form a disulfide bond (e.g. Serine, Alanine, Glycine) with an amino acid residue capable of forming a disulfide bond (e.g. cysteine), the insertion of one or more amino acid residues which increase the rigidity of the polypeptide (e.g., Proline) and the deletion of one or more amino acid residues which increase the flexibility (e.g., Glycine).

In a specific embodiment, the engineered antibodies disclosed herein comprise a modified hinge having altered the hinge conformation relative to a wild type hinge. A modified hinge having altered hinge conformation can be generated by incorporating certain modifications into a wild type hinge. Hinge modifications which alter the conformation of the hinge include, but are not limited to, the substitution of one or more amino acids residues with small side chains (e.g., alanine, glycine) for those with larger more bulky side chains (e.g., tryptophan, proline), the substitution of one or more amino acids residues with larger more bulky side chains (e.g., tryptophan, proline) for those with small side chains (e.g., alanine, glycine), the inversion of two or more amino acid resides within the hinge, the insertion or deletion of one or more amino acid residues with large or bulky side chains (e.g., tryptophan, proline). In addition, hinge modifications which alter the length and/or flexibility of the hinge may also result in an alteration of the conformation.

In a specific embodiment, the engineered antibodies disclosed herein comprise a modified hinge having an IgG2a camel-like modification. A modified hinge having a camel-like modification may be generated by substituting a portion of the wild type hinge with a portion of a camel IgG2a hinge. Alternatively, or optionally, a modified hinge having a camel-like modification can be generated by substituting one or more amino acid residue in the hinge with the corresponding amino acid residue found in the camel IgG2a hinge. A modified hinge having a camel-like modification can also incorporate additional amino acid substitutions and/or insertions and/or deletions. In addition, a camel-like modification of the hinge can alter characteristics of the hinge including but not limited to, the length, the flexibility, the conformation, the charge, and the hydrophobicity.

In another embodiment, the engineered antibodies disclosed herein comprise a modified hinge having altered charge relative to a wild type hinge. A modified hinge having altered charge can be generated by incorporating certain modifications into a wild type hinge. Hinge modifications which alter the charge of the hinge include but are not limited to, the substitution of one or more amino acids residues with a neutral charge (e.g., valine, threonine) for those with a charge (e.g., aspartate, glutamate, lysine, arginine), the substitution of one or more amino acid residues with a positive charge (e.g., lysine, arginine) for those with a neutral (e.g., valine, threonine) or negative charge (e.g., aspartate, glutamate), the substitution of one or more amino acid residues with a negative charge (e.g., aspartate, glutamate) for those with a neutral (e.g., valine, threonine) or positive charge (e.g., lysine, arginine) and the insertion or deletion of one or more charged amino acid residues (e.g., aspartate, glutamate, lysine, arginine).

In another specific embodiment, the engineered antibodies disclosed herein comprise a modified hinge having altered (e.g., increased or decreased) hydrophobicity relative to a wild type hinge. A modified hinge having altered hydrophobicity can be generated by incorporating certain modifications into a wild type hinge. Hinge modifications which alter the hydrophobicity of the hinge include but are not limited to, the substitution of one or more hydrophobic amino acids residues (e.g., valine, leucine) for hydrophilic amino acid residues (e.g., serine, threonine, tyrosine), the substitution of one or more hydrophilic amino acid (e.g., serine, threonine, tyrosine), for hydrophobic amino acids residues (e.g., valine, leucine), and the insertion or deletion of one or more hydrophobic or hydrophilic amino acid residues (e.g., valine, leucine, serine, threonine, tyrosine).

It will be recognized by one of skill in the art that any given hinge modification may alter more than one characteristic of the hinge. For example, the addition of one or more proline residue into the hinge results in a hinge modification that increases the length of the hinge while at the same time potentially decreasing the flexibility. Likewise, the substitution of a glycine residue with an aspartate can alter both the charge and the hydrophobicity of the hinge. Other combinations are described above and still others will be apparent to one skilled in the art.

It is specifically contemplated that one may choose to analyze the nature of the amino acid residues present in the hinge prior to making any hinge modifications. One skilled in the art will appreciate that in some cases an antibody of interest will already have the appropriate amino acid sequence within the hinge such that one or more characteristic (e.g., flexibility, length, conformation, charge and hydrophobicity) of the hinge is altered compared to a wild type hinge. In this situation, additional hinge modifications will only be introduced if further modification is desirable. It will be apparent to one skilled in the art that in addition to the specific amino acid residues described above and listed in Table 1, a number of additional amino acid residues may be inserted, deleted and/or substituted in the hinge to change the characteristics of the hinge. Families of amino acid residues having similar properties have been defined in the art and several examples are shown in Table 2.

TABLE 2
Properties of amino acid residues

| Family | Amino Acids |
|---|---|
| non-polar (hydrophobic) | Trp, Phe, Met, Leu, Ile, Val, |
| | Ala, Pro, Gly, |
| uncharged polar (hydrophillic) | Ser, Thr, Asa, Glu, Tyr, Cys |
| acidic/negatively charged | Asp, Gln |
| basic/positively charged | Arg, Lys, His |
| Bata-branched | Thr, Val, Ile |
| residues that influence | Gly, Pro |
| chain orientation | |
| aromatic | Trp, Tyr, Phe, His |

It is specifically contemplated that conservative amino acid substitutions may be made for said modifications of the hinge, described supra. It is well known in the art that “conservative amino acid substitution” refers to amino acid substitutions that substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in Table 2 (supra).

The term “conservative amino acid substitution” also refers to the use of amino acid analogs or variants. Guidance concerning how to make phenotypically silent amino acid substitutions is provided in Bowie et al., “Deciphering the Message in Protein Sequences: Tolerance to Amino Acid Substitutions,” (1990, Science 247:1306-1310).

Stability of the engineered antibodies disclosed herein can be examined by measuring a variety of different characteristics of a polypeptide which can alter its biological function and/or activity. Non-limiting examples of such characteristics include aggregation, fragmentation, the presence or absence of protein modifications (e.g., acetylation, glycosylation, methylation, phosphorylation), biological activity and dissociation of multi-subunit complexes. Generally, one or more of these characteristics is monitored (i.e., measured) over a period of time under a set of pre-determined conditions. The stability of the disclosed engineered antibodies may be characterized using in vitro stability assays known in the art for determining the stability of a polypeptide. Such assays include, but are not limited to, monitoring the integrity of the polypeptide over time using assays that monitor the size and/or activity of the polypeptide. The stability engineered antibodies can be examined in solution or as a solid. In addition, the stability can be monitored in the presence of components known to affect the stability of antibodies such as, but not limited to, metal ions and proteases.

In one embodiment, the engineered antibodies disclosed herein are antibodies or Fc fusion proteins comprising a modified hinge, wherein said modified hinge improves stability or is resistant to cleave or is less prone to cleavage relative to a comparable molecule that lacks a modified hinge. Such antibodies include IgG molecules containing a hinge which can be modified to generate a modified hinge. As such, the engineered antibodies disclosed herein can include any antibody molecule that binds, preferably, specifically (i.e., competes off non-specific binding as determined by immunoassays well known in the art for assaying specific antigen-antibody binding) an antigen incorporating a modified hinge. Such antibodies include, but are not limited to, polyclonal, monoclonal, bi-specific, multi-specific, human, humanized, chimeric antibodies, single chain antibodies, Fab fragments, F(ab′)2 fragments, disulfide-linked Fvs, and fragments containing either a VL or VH domain or even a complementary determining region (CDR) that specifically binds an antigen, in certain cases, engineered to contain or fused to a hinge-region containing polypeptide.

Also disclosed herein are engineered antibodies with improved stability. In certain embodiments, antibodies comprising modified hinge regions are more resistant to cleavage then a comparable molecule. In a specific embodiment, the engineered antibodies are more resistant to metal ion-mediated cleavage, in particular cation ion-mediated cleavage in the hinge. In one embodiment, engineered antibodies are at least 2 fold, or at least 3 fold, or at least 5 fold, or at least 10 fold, or at least 50 fold, or at least 100 fold more resistant to cleavage than a comparable molecule lacking a modified hinge region. In another embodiment, the engineered antibodies disclosed herein are at least 10%, or at least 20%, or at least 30%, or at least 40%, or at least 50%, or at least 60%, or at least 70%, or at least 80%, or at least 90%, or at least 100%, or at least 150%, or at least 200% more resistant to cleavage than a comparable molecule lacking a modified hinge region.

It is contemplated that the engineered antibodies disclosed herein can have other altered characteristics including increased in vivo half-lives (e.g., serum half-lives) in a mammal; in particular, a human, increased stability in vivo (e.g., serum half-lives) and/or in vitro (e.g., shelf-life) and/or increased melting temperature (Tm), relative to a comparable molecule. In one embodiment, an engineered antibody disclosed herein has an in vivo half-life of greater than 15 days, greater than 20 days, greater than 25 days, greater than 30 days, greater than 35 days, greater than 40 days, greater than 45 days, greater than 2 months, greater than 3 months, greater than 4 months, or greater than 5 months. In another embodiment, an engineered antibody has an in vitro half-live (e.g., liquid or powder formulation) of greater than 15 days, greater than 30 days, greater than 2 months, greater than 3 months, greater than 6 months, or greater than 12 months, or greater than 24 months, or greater than 36 months, or greater than 60 months. In still another embodiment, an engineered antibody has a Tm value higher than about 60° C., 65° C., 70° C., 75° C., 80° C., 85° C., 90° C., or 95° C.

It is also specifically contemplated that the engineered antibodies disclosed herein can contain inter alia one or more additional amino acid residue substitutions, mutations and/or modifications which result in an antibody with preferred characteristics including, but not limited to: increased serum half-life, increase binding affinity, reduced immunogenicity, increased production, enhanced or reduced ADCC or CDC activity, altered glycosylation and/or disulfide bonds and modified binding specificity.

The engineered antibodies disclosed herein can be combined with other modifications, including but not limited to, modifications that alter effector function. For example, combining an engineered antibody disclosed herein with other modifications to provide additive, synergistic, or novel properties in antibodies or fusions. Such modifications can be in the CH1, CH2, or CH3 domains or a combination thereof. It is contemplated that the engineered antibodies enhance the property of the modification with which they are combined. For example, if an engineered antibody (such as any of the engineered antibodies with modified hinge regions disclosed herein) is combined with a mutant known to bind FcγRIIIA with a higher affinity than a comparable molecule comprising a wild type Fc region; the combination with a mutant results in a greater fold enhancement in FcγRIIIA affinity.

In some embodiments, the engineered antibodies disclosed herein comprise one or more engineered glycoforms, i.e., a carbohydrate composition that is covalently attached to a molecule comprising an engineered hinge region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example B (1,4)-N-acetylglucosaminyltransferase III (GnTI11), by expressing a molecule comprising a modified hinge region in various organisms or cell lines from various organisms, or by modifying carbohydrate(s) after the molecule comprising a modified hinge region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US20030115614; Okazaki et al., 2004, JMB, 336:1239-49.

It is contemplated that the engineered antibodies disclosed herein include antibodies comprising a variable region, an Fc region, and a modified hinge (such as any of the modified hinge regions disclosed herein). The engineered antibodies can be produced “de novo” by combing a variable domain, of fragment thereof, that specifically binds at least one antigen with an Fc region incorporating a modified hinge. Alternatively, engineered antibodies can be produced by modifying the hinge of an Fc region-containing antibody that binds an antigen.

Antibody types contemplated for use with the modified hinge regions disclosed herein can include, but are not limited to, synthetic antibodies, monoclonal antibodies, recombinantly produced antibodies, intrabodies, multispecific antibodies, bispecific antibodies, human antibodies, humanized antibodies, chimeric antibodies, synthetic antibodies, single-chain FvFcs (scFvFc), single-chain Fvs (scFv), and anti-idiotypic (anti-Id) antibodies. In particular, antibodies used in the methods disclosed herein include immunoglobulin molecules and immunologically active portions of immunoglobulin molecules. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule.

The engineered antibodies disclosed herein can be derived from any animal origin including birds and mammals (e.g., human, murine, donkey, sheep, rabbit, goat, guinea pig, camel, horse, or chicken). In a specific embodiment, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies disclosed herein can be monospecific, bispecific, trispecific or have greater multispecificity. Multispecific antibodies may specifically bind to different epitopes of desired target molecule or may specifically bind to both the target molecule as well as a heterologous epitope, such as a heterologous polypeptide or solid support material. See, e.g., International Publication Nos. WO 94/04690; WO 93/17715; WO 92/08802; WO 91/00360; and WO 92/05793; Tutt, et al., 1991, J. Immmmol. 147:60-69; U.S. Pat. Nos. 4,474,893, 4,714,681, 4,925,648, 5,573,920, and 5,601,819; and Kostelny et al., 1992, J. Immunol. 148:1547). Antibodies with more than two valencies incorporating the modified hinge disclosed herein are contemplated. For example, trispecific antibodies can be prepared. See, e.g., Tutt et al. J. Immunol. 147:60 (1991).

Engineered antibodies contemplated herein also encompass single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079).

The engineered antibodies disclosed herein can further encompasses antibody-like and antibody-domain fusion proteins. An antibody-like molecule is any molecule that has been generated with a desired binding property, see, e.g., PCT Publication Nos. WO 04/044011; WO 04/058821; WO 04/003019 and WO 03/002609. Antibody-domain fusion proteins may incorporate one or more antibody domains such as the Fc domain or the variable domain. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof. A large number of antibody-domain molecules are known in the art including, but not limited to, diabodies (dsFv)₂ (Bera et al., 1998, J. Mol. Biol. 281:475-83); minibodies (homodimers of scFv-CH3 fusion proteins) (Pessi et al., 1993, Nature 362:367-9), tetravalent di-diabody (Lu et al., 2003 J. Immunol. Methods 279:219-32), tetravalent bi-specific antibodies called Bs(scFv)4-IgG (Zuo et al., 2000, Protein Eng. 13:361-367). Fc domain fusions combine the Fc region of an immunoglobulin, specifically an Fc region comprising a modified hinge, with a fusion partner which in general can be a protein, including, but not limited to, a ligand, an enzyme, the ligand portion of a receptor, an adhesion protein, or some other protein or domain. See, e.g., Chamow et al., 1996, Trends Biotechnol 14:52-60; Ashkenazi et al., 1997, Curr Opin Immunol 9:195-200; Heidaran et al., 1995, FASEB J. 9:140-5. Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; PCT Publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Other molecules specifically contemplated are small, engineered protein domains such as, for example, immuno-domains and/or monomer domains (see for example, U.S. Patent Publication Nos. 2003082630 and 2003157561). Immuno-domains contain at least one complementarity determining region (CDR) of an antibody while monomer domains are based upon known naturally-occurring, non-antibody domain families, specifically protein extracellular domains, which contain conserved scaffold and variable binding sites, an example is the LDL receptor A domain which is involved in ligand binding. Such protein domains can correctly fold independently or with limited assistance from, for example, a chaperonin or the presence of a metal ion. This ability avoids mis-folding of the domain when it is inserted into a new protein environment, thereby preserving the protein domain's binding affinity for a particular target. The variable binding sites of the protein domains are randomized using various diversity generation methods such as, for example, random mutagenesis, site-specific mutagenesis, as well as by directed evolution methods, such as, for example, recursive error-prone PCR, recursive recombination and the like. For details of various diversity generation methods see U.S. Pat. Nos. 5,811,238; 5,830,721; 5,834,252; PCT Publication Nos. WO 95/22625; WO 96/33207; WO 97/20078; WO 97/35966; WO 99/41368; WO 99/23107; WO 00/00632; WO 00/42561; and WO 01/23401. The mutagenized protein domains are then expressed using a display system such as, for example, phage display, which can generate a library of at least 1010 variants and facilitate isolation of those protein domains with improved affinity and potency for an intended target by subsequent panning and screening. Such methods are described in PCT publication Nos. WO 91/17271; WO 91/18980; WO 91/19818; WO 93/08278. Examples of additional display systems are described in U.S. Pat. Nos. 6,281,344; 6,194,550; 6,207,446; 6,214,553 and 6,258,558. Utilizing these methods, a high diversity of engineered protein domains having sub-nM binding affinity (Kd) and blocking function (IC50) can be rapidly generated. Once identified two to ten such engineered protein domains can be linked together, using natural protein linkers of about 4-15 amino acids in length, to form a binding protein. The individual domains can target a single type of protein or several, depending upon the use/disease indication. The engineered protein domains can then be linked to an Fc region in an antibody containing a modified hinge, such as any of the modified hinge regions disclosed herein.

The introduction of a modified hinge (such as any of the modified hinges disclosed herein) into an antibody already described in the art is also contemplated. Antibodies into which a modified hinge is introduced may specifically bind a cancer or tumor antigen for example, including, but not limited to, KS 1/4 pan-carcinoma antigen (Perez and Walker, 1990, J. Immunol. 142:3662-3667; Bumal, 1988, Hybridoma 7(4): 407-415), ovarian carcinoma antigen (CA125) (Yu et al., 1991, Cancer Res. 51(2): 468-475), prostatic acid phosphate (Tailor et al., 1990, Nucl. Acids Res. 18(16): 4928), prostate specific antigen (Henttu and Vihko, 1989, Biochem. Biophys. Res. Comm. 160(2): 903-910; Israeli et al., 1993, Cancer Res. 53:227-230), melanoma-associated antigen p97 (Estin et al., 1989, J. Natl. Cancer Instil. 81(6): 445-446), melanoma antigen gp75 (Vijayasardahl et al., 1990, J. Exp. Med. 171(4): 1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185HER2), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm I antigen found in adult erythrocytes, preimplantation embryos, I (Ma) found in gastric adenocarcinomas, M18, M39 found in breast epithelium, SSEA-1 found in myeloid cells, VEP8, VEP9, Myl, VIM-D5, D156-22 found in colorectal cancer, TRA-1-85 (blood group H), C14 found in colonic adenocarcinoma, F3 found in lung adenocarcinoma, AH6 found in gastric cancer, Y hapten, Lc^{y} found in embryonal carcinoma cells, TLS (blood group A), EGF receptor found in A431 cells, E1 series (blood group B) found in pancreatic cancer, FC 10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{v}) found in colonic adenocarcinoma, 19.9 found in colon cancer, gastric cancer mucins, T₅A₇ found in myeloid cells, R24 found in melanoma, 4.2, G_{D3}, D1.1, OFA-1, G_{M2}, OFA-2, G_{D2}, and M1: 22:25:8 found in embryonal carcinoma cells, and SSEA-3 and SSEA-4 found in 4 to 8-cell stage embryos. In one embodiment, the antigen is a T cell receptor derived peptide from a Cutaneous Tcell Lymphoma (see, Edelson, 1998, The Cancer Journal 4:62).

In some embodiments, a modified hinge can be introduced into an anti-fluoresceine monoclonal antibody, Apr. 4, 2020 (Kranz et al., 1982 J. Biol. Chem. 257(12): 6987-6995). In other embodiments, a modified hinge is introduced into a mouse-human chimeric anti-CD20 monoclonal antibody 2H7, which recognizes the CD20 cell surface phosphoprotein on B cells (Liu et al., 1987, Journal of Immunology, 139:3521-6). In yet other embodiments, a modified hinge is introduced into a humanized antibody (Ab4D5) against the human epidermal growth factor receptor 2 (p185 HER2) as described by Carter et al. (1992, Proc. Natl. Acad. Sci. USA 89:4285-9). In yet other embodiments a modified hinge is introduced into a humanized anti-TAG72 antibody (CC49) (Sha et al., 1994 Cancer Biother. 9(4): 341-9). In other embodiments, modified hinge is introduced into Rituxan which is used for treating lymphomas.

In some embodiments, a modified hinge (such as any of the modified hinges imparting resistance to proteolytic cleavage disclosed herein) can be introduced into a therapeutic monoclonal antibody specific for a cancer antigen or cell surface receptor including but not limited to, Erbitux™ (also known as IMC-C225) (ImClone Systems Inc.), a chimerized monoclonal antibody against EGFR; HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD18 F(ab′)₂ (Genentech); CDP860 which is a humanized anti-CD18 F(ab′)₂ (Celltech, UK); PRO542 which is an anti-HIV gp120 antibody fused with CD4 (Progenics/Genzyme Transgenics); C14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DR antibody (Techniclone); anti-CD11a is a humanized IgG1 antibody (Genetech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); IDEC-151 is a primatized anti-CD4 IgG1 antibody (IDEC Pharm/SmithKline Beecham); MDX-CD4 is a human anti-CD4

IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); rhuMab-E25 is a humanized anti-IgE IgG1 antibody (Genentech/Norvartis/Tanox Biosystems); IDEC-152 is a primatized anti-CD23 antibody (IDEC Pharm); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β2-integrin IgG antibody (LeukoSite); Anti-LFA-1 is a murine anti CD18 F(ab′)₂ (Pasteur-Merieux/Immunotech); CAT-152 is a human anti-TGF-β₂ antibody (Cambridge Ab Tech); and Corsevin M is a chimeric anti-Factor VII antibody (Centocor).

In some embodiments, the engineered antibody or functional fragment thereof is an anti-Respiratory Syncytial Virus (RSV) antibody, an anti-ebola virus antibody, an anti-aggregated β-amyloid (AB) antibody, an anti-human immunodeficiency virus (HIV) antibody, an anti-herpes simplex virus (HSV) antibody, an anti-sperm antibody (such as an anti-human contraceptive antigen (HCA) antibody), and anti-HER2/neu antibody.

In still another embodiment, the engineered antibodies disclosed herein can specifically bind to the same antigen as a known therapeutic antibody including, but not limited to those listed supra, provided in some embodiments that the variable region of the engineered antibodies is not that of said therapeutic antibody. In other embodiments, the variable region of the engineered antibodies is identical to that of the therapeutic antibody.

### 1. Signal Sequences

In some embodiments, the engineered antibodies disclosed herein can include a signal sequence. The signal sequence can be any signal sequence that facilitates protein secretion from a host cell (e.g., a filamentous fungal host cell). In particular embodiments, the engineered antibody can comprise a signal sequence for a protein that is known to be highly secreted from a host cell in which the fusion protein is to be produced. The signal sequence employed can be endogenous or non-endogenous to the host cell in which the engineered antibody is to be produced.

Suitable signal sequences are known in the art (see, e.g., Ward et al, Bio/Technology 1990 8:435-440; and Paloheimo et al, Applied and Environmental Microbiology 2003 69:7073-7082). Non-limiting examples of suitable signal sequences include those of cellobiohydrolase I, cellobiohydrolase II, endoglucanases I, II and III, α-amylase, aspartyl proteases, glucoamylase, phytase, mannanase, α and β glucosidases, bovine chymosin, human interferon and human tissue plasminogen activator and synthetic consensus eukaryotic signal sequences such as those described by Gwynne et al., (1987) Bio/Technology 5:713-719.

In some embodiments, if Trichoderma (e.g. T. reesei) is employed as a host cell, the signal sequence or carrier of T. reesei mannanase I (Man5A, or MANI), T. reesei cellobiohydrolase II (Cel6A or CBHII), endoglucanase I (Cel7b or EGI), endoglucanase II (Cel5a or EGII), endoglucanase III (Cel12A or EGIII), xylanases I or II (XynIIa or XynIIb) or T. reesei cellobiohydrolase I (Cel7a or CBHI) can be employed in the engineered antibody.

In other embodiments, if an Aspergillus (e.g. A. niger) is employed as a host cell, the signal sequence or carrier of A. niger glucoamylase (GlaA) or alpha amylase can be employed in the fusion polypeptide. Aspergillus niger and Aspergillus awamori glucoamylases have identical amino acid sequences. Two forms of the enzyme are generally recognized in culture supernatants. GAI is the full-length form (amino acid residues 1-616) and GAII is a natural proteolytic fragment comprising amino acid residues 1-512. GAI is known to fold as two separate domains joined by an extended linker region. The two domains are the 471-residue catalytic domain (amino acids 1-471) and the 108 residue starch binding domain (amino acids 509-616), the linker region between the two domains being 36 residues (amino acids 472-508). GAII lacks the starch binding domain. Reference is made to Libby et al., (1994) Protein Engineering 7:1109-1114. In some embodiments, the glucoamylase which is used as a carrier protein and including a signal sequence will have greater than 95%, 96%, 97%, 98% and 99% sequence identity with a catalytic domain of an Aspergillus or Trichoderma glucoamylase. The term “catalytic domain” refers to a structural portion or region of the amino acid sequence of a protein which possess the catalytic activity of the protein.

### 2. Carriers

In particular embodiments, the signal sequence can comprise a “carrier” that contains the signal sequence at its N-terminus, where the carrier is at least an N-terminal portion of a protein that is endogenous to the cell and efficiently secreted by a cell. In certain embodiments, the signal sequence and the carrier protein are obtained from the same gene. In some embodiments, the signal sequence and the carrier protein are obtained from different genes.

The carrier protein can include all or part of the mature sequence of a secreted polypeptide. In some embodiments, full length secreted polypeptides are used. However, functional portions of secreted polypeptides can be employed. As used herein “portion” of a secreted polypeptide or grammatical equivalents means a truncated secreted polypeptide that retains its ability to fold into a normal, albeit truncated, configuration.

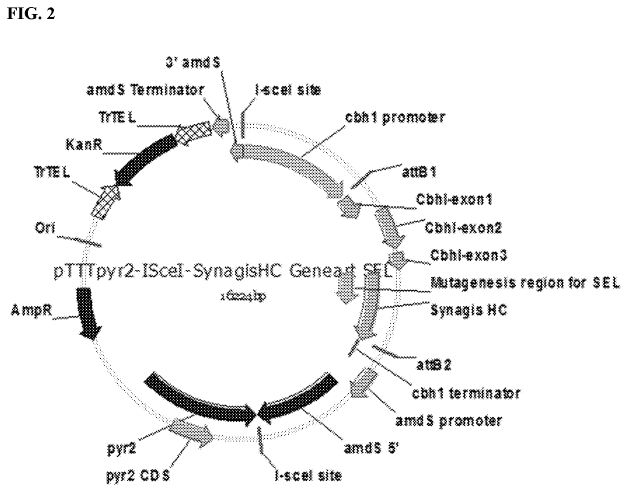

In some cases, the truncation of the secreted polypeptide means that the functional protein retains a biological function. In some embodiments, the catalytic domain of the secreted polypeptide is used, although other functional domains could be used, for example the substrate binding domain. In one embodiment, when glucoamylase is used as the carrier protein (e.g. glucoamylase from Aspergillus niger), functional portions retain the catalytic domain of the enzyme and include amino acids 1-471 (see, WO 03089614, e.g., Example 10, the disclosure of which is incorporated by reference herein). In another embodiment, when CBH I is used as the carrier protein (i.e. CBH I from Trichoderma reesei) functional portions retain the catalytic domain of the enzyme. Reference is made to SEQ ID NO: 1 of FIG. 2 of WO 05093073, the disclosure of which is incorporated by reference herein, wherein the sequence encoding a Trichoderma reesei CBH1 signal sequence, T. reesei CBH1 catalytic domain (also referred to as catalytic core or core domain) and T. reesei CBH1 linker is disclosed. In some embodiments, a CBH1 carrier protein and including a signal sequence will have greater than 95%, 96%, 97%, 98% and 99% sequence identity with SEQ ID NO: 1 of FIG. 2 of WO 05093073, the disclosure of which is incorporated by reference herein).

In general, if the carrier protein is a truncated protein, it is C-terminally truncated (i.e., contains an intact N-terminus). Alternatively, the carrier protein can be N-terminally truncated, or optionally truncated at both ends to leave a functional portion. Generally, such portions of a secreted protein which comprise a carrier protein comprise greater than 50%, greater than 70%, greater than 80% and greater than 90% of the secreted protein and, in some embodiments, the N-terminal portion of the secreted protein. In some embodiments, the carrier protein will include a linker region in addition to the catalytic domain. In some embodiments, a portion of the linker region of the CBHI protein can be used in the carrier protein.

In some embodiments, the first amino acid sequence comprising a signal sequence functional as a secretory sequence is encoded by a first DNA molecule. The second amino acid sequence comprising the carrier protein is encoded by a second DNA sequence. However, as described above the signal sequence and the carrier protein can be obtained from the same gene.

### 3. Antibody Conjugates and Derivatives

Any of the engineered antibodies disclosed herein can include derivatives that are modified (i.e., by the covalent attachment of any type of molecule to the antibody such that covalent attachment). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

Further, antibodies can be conjugated to albumin in order to make the antibody or antibody fragment more stable in vivo or have a longer half-life in vivo. The techniques are well known in the art, see e.g., International Publication Nos. WO 93/15199, WO 93/15200, and WO 01/77137; and European Patent No. EP 413, 622. The present invention encompasses the use of antibodies or fragments thereof conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses the use of antibodies or fragments thereof recombinantly fused or chemically conjugated (including both covalent and non-covalent conjugations) to a heterologous protein or polypeptide (or fragment thereof, for example, to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

The present invention further includes compositions comprising heterologous proteins, peptides or polypeptides fused or conjugated to antibody fragments. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Additional fusion proteins, e.g., of antibodies that specifically bind an antigen (e.g., supra), may be generated through the techniques of gene-shuffling, motif-shuffling, exon-shuffling, and/or codon-shuffling (collectively referred to as “DNA shuffling”). DNA shuffling may be employed to alter the activities of the engineered antibodies disclosed herein or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2): 76-82; Hansson, et al., 1999, J. Mol. Biol. 287:265-76; and Lorenzo and Blasco, 1998, Biotechniques 24(2): 308-313. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to an Antigen may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, the engineered antibodies disclosed herein or analogs or derivatives thereof are conjugated to a diagnostic or detectable agent. Such antibodies can be useful for monitoring or prognosing the development or progression of a cancer as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I) carbon (¹⁴C), sulfur (³⁵S), tritium (³H), indium ¹¹⁵In, ¹¹³In, ¹¹²In, ¹¹¹In,), and and technetium (⁹⁹Tc), thallium (²⁰¹Ti), gallium (⁶⁸Ga, ⁶⁷Ga), palladium (¹⁰³Pd), molybdenum (⁹⁹Mo), xenon (₁₃₃Xe), fluorine (¹⁸F), ¹⁵³Sm, ¹⁷⁷Lu, ¹³⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

Use of the engineered antibodies disclosed herein or fragments thereof conjugated to a therapeutic agent is also contemplated. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include ribonuclease, monomethylauristatin E and F, paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A more extensive list of therapeutic moieties can be found in PCT publications WO 03/075957, incorporated by reference herein.

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

Techniques for conjugating therapeutic moieties to antibodies and related molecules are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119.

Methods for fusing or conjugating antibodies and related molecules to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337. The fusion of an antibody to a moiety does not necessarily need to be direct, but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an engineered antibody (such as any of those disclosed herein) should be chosen to achieve the desired prophylactic or therapeutic effect(s) for a particular disorder in a subject. A clinician or other medical personnel should consider the following when deciding on which therapeutic moiety or drug to conjugate to an engineered antibody: the nature of the disease, the severity of the disease, and the condition of the subject.

### B. Polynucleotides

Another aspect of the compositions and methods disclosed herein is a polynucleotide or a nucleic acid sequence that encodes an engineered antibody, such as any of the engineered antibodies disclosed herein.

A fusion DNA construct encoding an engineered antibody as disclosed above is provided herein, comprising in operable linkage a promoter; a first DNA molecule encoding a signal sequence; a second DNA molecule encoding a carrier protein; a third DNA molecule encoding an antibody (e.g. a heavy chain and/or a light chain) or functional fragment thereof. The components of the fusion DNA construct can occur in any order. Since the genetic code is known, the design and production of these nucleic acids is well within the skill of an ordinarily skilled artisan, given the description of the engineered antibodies disclosed herein. In certain embodiments, the nucleic acids can be codon optimized for expression of the engineered antibodies in a particular host cell. Since codon usage tables are available for many species of, for example, mammalian cells and filamentous fungi, the design and production of codon-optimized nucleic acids that encodes subject engineered antibodies would be well within the skill of one of skill in the art.

### C. Promoters

Examples of suitable promoters for directing the transcription of a nucleic acid in a host cell (for example, a filamentous fungal host cell) are promoters obtained from the genes for Aspergillus oryzae TAKA amylase, Rhizomucor miehei aspartic proteinase, Aspergillus niger neutral alpha-amylase, Aspergillus niger acid stable alpha-amylase (Korman et al (1990) Curr. Genet 17:203-212; Gines et al., (1989) Gene 79:107-117), Aspergillus niger or Aspergillus awamori glucoamylase (glaA) (Nunberg et al., (1984) Mol. Cell Biol. 4:2306-2315; Boel E. et al., (1984) EMBO J. 3:1581-1585), Rhizomucor miehei lipase, Aspergillus oryzae alkaline protease, Aspergillus oryzae triose phosphate isomerase, Aspergillus nidulans acetamidase (Hyner et al., (1983) Mol. Cell. Biol. 3:1430-1439), Fusarium venenatum amyloglucosidase, Fusarium oxysporum trypsin-like protease (WO 96/00787), Trichoderma reesei cellobiohydrolase I (Shoemaker et al. (1984) EPA EPO 0137280), Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof. Reference is also made to Yelton et al., (1984) Proc. Natl. Acad. Sci. USA 81:1470-1474; Mullaney et al., (1985) Mol. Gen. Genet. 199:37-45; Lockington et al., (1986) Gene 33:137-149; Macknight et al., (1986) Cell 46:143-147; Hynes et al., (1983) Mol. Cell Biol. 3:1430-1439. Higher eukaryotic promoters such as SV40 early promoter (Barclay et al (1983) Molecular and Cellular Biology 3:2117-2130) can also be useful. Promoters can be constitutive or inducible promoters. Exemplary promoters include a Trichoderma reesei cellobiohydrolase I or II, a Trichoderma reesei endoglucanase I, II or III, and a Trichoderma reesei xylanase II.

### D. Vectors

A polynucleotide encoding any of the engineered antibodies disclosed herein can be present in a vector, for example, a phage, plasmid, viral, or retroviral vector. In certain embodiments, the vector can be an expression vector for expressing a subject fusion polypeptide in a filamentous fungal cell.

Vectors for expression of recombinant proteins are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

A fusion DNA construct can be constructed using well known techniques as is generally described for example in European Patent Application Publication No. 0 215 594, the disclosure of which is incorporated by reference herein.

Natural or synthetic polynucleotide fragments encoding for the polypeptide of interest (e.g. an immunoglobulin) can be incorporated into heterologous nucleic acid constructs or vectors, capable of introduction into and replication in a host cell (e.g., a filamentous fungal host cell).

Once a DNA construct or more specifically a fusion DNA construct is made it can be incorporated into any number of vectors as is known in the art. While the DNA construct will in some embodiments include a promoter sequence, in other embodiments the vector will include other regulatory sequences functional in the host to be transformed, such as ribosomal binding sites, transcription start and stop sequences, terminator sequences, polyadenylation signals, enhancers and or activators. In some embodiments, a polynucleotide encoding engineered antibodies is inserted into a vector which comprises a promoter, signal sequence and carrier protein at an appropriate restriction endonuclease site by standard procedures. Such procedures and related sub-cloning procedures are deemed to be within the scope of knowledge of those skilled in the art.

Terminator sequences which are recognized by the expression host to terminate transcription can be operably linked to the 3′ end of the fusion DNA construct encoding the engineered antibodies to be expressed. Those of general skill in the art are well aware of various terminator sequences that can be used with host cells, such as, filamentous fungi. Non-limiting examples include the terminator from the Aspergillus nidulans trpC gene (Yelton M. et al., (1984) Proc. Natl. Acad. Sci. USA 81:1470-1474) or the terminator from the Aspergillus niger glucoamylase genes (Nunberg et al. (1984) Mol. Cell. Biol. 4:2306-2353) or the terminator from the Trichoderma reesei cellobiohydrolase I gene.

Polyadenylation sequences are DNA sequences which when transcribed are recognized by the expression host to add polyadenosine residues to transcribed mRNA. Examples include polyadenylation sequences from A. nidulans trpC gene (Yelton et al (1984) Proc. Natl. Acad. Sci. USA 81; 1470-1474); from A. niger glucoamylase gene (Nunberg et al. (1984) Mol. Cell. Biol. 4:2306-2315); the A. oryzae or A. niger alpha amylase gene and the Rhizomucor miehei carboxyl protease gene.

In further embodiments, the fusion DNA construct or the vector comprising the fusion DNA construct will contain a selectable marker gene to allow the selection of transformed host cells. Selection marker genes are well known in the art and will vary with the host cell used. Examples of selectable markers include but are not limited to ones that confer antimicrobial resistance (e.g. hygromycin, bleomycin, chloroamphenicol and phleomycin). Genes that confer metabolic advantage, such as nutritional selective markers can also find use. Some of these markers include amdS. Also, sequences encoding genes which complement an auxotrophic defect can be used as selection markers (e.g. pyr4 complementation of a pyr4 deficient A. nidulans, A. awamori or Trichoderma reesei and argB complementation of an argB deficient strain). Reference is made to Kelley et al., (1985) EMBO J. 4:475-479; Penttila et al., (1987) Gene 61:155-164 and Kinghorn et al (1992) Applied Molecular Genetics of Filamentous Fungi, Blackie Academic and Professional, Chapman and Hall, London, the disclosure of each of which are incorporated by reference herein.

### E. Host Cells

The expression cassette or vector can be introduced into a suitable expression host cell, which then expresses the corresponding polynucleotide encoding an engineered antibody.

Suitable host cells include cells of any microorganism (e.g., cells of a bacterium, a protist, an alga, a fungus (e.g., a yeast or filamentous fungus), or other microbe), and can be cells of a bacterium, a yeast, or a filamentous fungus. Fungal expression hosts can be, for example, yeasts, which can also serve as ethanologens. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing the polypeptide.

Suitable host cells of the bacterial genera include, but are not limited to, cells of Escherichia, Proteus, Bacillus, Ralstonia, Lactobacillus, Lactococcus, Pseudomonas, Staphylococcus, and Streptomyces. Suitable cells of bacterial species include, but are not limited to, cells of Escherichia coli, Bacillus subtilis, Bacillus licheniformis, Bacillus megaterium, Lactobacillus brevis, Pseudomonas aeruginosa, Pseudomonas fluorescens, Pseudomonas stutzerei, Staphylococcus carnosus, Lactococcus lactis, Ralstonia eutropha, Proteus mirabilis, and Streptomyces lividans.

Suitable host cells of the genera of yeast include, but are not limited to, cells of Saccharomyces, Schizosaccharomyces, Candida, Hansenula, Pichia, Kluyveromyces, Yarrowia and Phaffia. Suitable cells of yeast species include, but are not limited to, cells of Saccharomyces cerevisiae, Schizosaccharomyces pombe, Candida albicans, Hansenula polymorpha, Yarrowia lipolytica, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Promoters and/or signal sequences associated with secreted proteins in a particular host of interest are candidates for use in the heterologous production and secretion of engineered antibodies in that host or in other hosts. As a non-limiting example, in filamentous fungal systems, the promoters that drive the genes for cellobiohydrolase I (cbh1), glucoamylase A (glaA), TAKA-amylase (amyA), xylanase (exlA), the gpdA-promoter cbh1, cbhll, endoglucanase genes eg1-eg5, Cel61B, Cel74A, gpd promoter, Pgk1, pki1, EF-1alpha, tef1, cDNA1 and hex1 are suitable and can be derived from a number of different organisms (e.g., A. niger, T. reesei, A. oryzae, A. awamori, A. nidulans).

In some embodiments, the polynucleotide encoding an engineered antibody is recombinantly associated with a polynucleotide encoding a suitable homologous or heterologous signal sequence that leads to secretion of the recombinant polypeptide into the extracellular (or periplasmic) space, thereby allowing direct detection in the cell supernatant (or periplasmic space or lysate). Suitable signal sequences for Escherichia coli, other gram-negative bacteria and other organisms known in the art include those that drive expression of the HlyA, DsbA, Pbp, PhoA, PelB, OmpA, OmpT or M13 phage Gill genes. For Bacillus subtilis, Gram-positive organisms and other organisms known in the art, suitable signal sequences further include those that drive expression of the AprE, NprB, Mpr, AmyA, AmyE, Blac, SacB, and for S. cerevisiae or other yeast, including the killer toxin, Bar1, Suc2, Mating factor alpha, Inu1A or Ggplp signal sequence. Signal sequences can be cleaved by a number of signal peptidases, thus removing them from the rest of the expressed protein.

In some embodiments, the engineered antibodies are expressed alone or as a fusion with additional peptides, tags or proteins located at the N- or C-terminus (e.g., 6×His, HA or FLAG tags). Suitable fusions include tags, peptides or proteins that facilitate affinity purification or detection (e.g., 6×His, HA, chitin binding protein, thioredoxin or FLAG tags), as well as those that facilitate expression, secretion or processing of the target beta-glucosidases. In addition to KEX2, further suitable processing sites include enterokinase, STE13, or other protease cleavage sites known in the art for cleavage in vivo or in vitro.

Polynucleotides encoding engineered antibodies can be introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

### III. Methods

### A. Methods for Generating Antibodies

The engineered antibodies disclosed herein (i.e., antibodies incorporating a modified hinge as described supra) can be produced by any method known in the art for the synthesis of antibodies, in particular, by chemical synthesis or by recombinant expression techniques.

Polyclonal antibodies recognizing a particular antigen can be produced by various procedures well known in the art. For example, an antigen or immunogenic fragments thereof can be administered to various host animals including, but not limited to, rabbits, mice, rats, etc. to induce the production of sera containing polyclonal antibodies specific for an antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an antigen or immunogenic fragment thereof and once an immune response is detected, e.g., antibodies specific for the administered antigen are detected in the mouse serum, the mouse spleen is harvested and splenocytes isolated. The splenocytes are then fused by well-known techniques to any suitable myeloma cells, for example cells from cell line SP20 available from the ATCC. Additionally, a RIMMS (repetitive immunization, multiple sites) technique can be used to immunize an animal (Kilpatrick et al., 1997, Hybridoma 16:381-9). Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody wherein, the hybridoma may be generated by fusing splenocytes isolated from a mouse immunized with an antigen or immunogenic fragments thereof, with myeloma cells and then screening the hybridomas resulting from the fusion for hybridoma clones that secrete an antibody able to bind the administered antigen.

The engineered antibodies disclosed herein can additionally contain novel amino acid residues in their hinge regions. Engineered antibodies can be generated by numerous methods well known to one skilled in the art. Non-limiting examples include, isolating antibody coding regions (e.g., from hybridoma) and introducing one or more hinge modifications of the invention into the isolated antibody coding region. Alternatively, the variable regions may be subcloned into a vector encoding comprising a modified hinge region (such as any of these disclosed herein). Additional methods and details are provided infra.

Antibody fragments that recognize specific an antigen can be generated by any technique known to those of skill in the art. For example, Fab and F(ab′)2 fragments of the invention can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the engineered antibodies disclosed herein can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an Antigen epitope of interest can be selected or identified with antigen, e.g., using labeled antigen or antigen bound or captured to a solid surface or bead. Examples of phage display methods that can be used to make the engineered antibodies disclosed herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6): 864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma constant, and the PCR amplified VL domains can be cloned into vectors expressing a VL constant region, e.g., human kappa or lamba constant regions. It is contemplated that the constant region comprises a modified hinge (such as any of the modified hinges disclosed herein). In certain embodiments, the vectors for expressing the VH or VL domains comprise a promoter, a secretion signal, a cloning site for both the variable and constant domains, as well as a selection marker such as neomycin. The VH and VL domains may also be cloned into one vector expressing the desired constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT Publication Nos. WO 98/46645, WO 98/50433, WO 98/24893, WO98/16654, WO 96/34096, WO 96/33735, and WO 91/10741.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. In a specific embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and lgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, or greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592, 106 and EP 519,596; Padlan, 1991, Molecular Immunology 28(4/5): 489-498; Studnicka et al., 1994, Protein Engineering 7(6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119-25 (2002), Caldas et al., Protein Eng. 13(5): 353-60 (2000), Morea et al., Methods 20(3): 267-79 (2000), Baca et al., J. Biol. Chem. 272(16): 10678-84 (1997), Roguska et al., Protein Eng. 9(10): 895-904 (1996), Couto et al., Cancer Res. 55 (23 Supp): 5973s-5977s (1995), Couto et al., Cancer Res. 55(8): 1717-22 (1995), Sandhu J S, Gene 150(2): 409-10 (1994), and Pedersen et al., J. Mol. Biol. 235(3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen or immunogenic fragments thereof. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598.

Further, the engineered antibodies disclosed herein can, in turn, be utilized to generate anti-idiotype antibodies that “mimic” a receptor using techniques well known to those skilled in the art. (See, e.g., Greenspan & Bona, 1989, FASEB J. 7(5): 437-444; and Nissinoff, 1991, J. Immunol. 147(8): 2429-2438). For example, antibodies of the invention which bind to and competitively inhibit the binding of a receptor (as determined by assays well known in the art and disclosed infra) to its ligands can be used to generate anti-idiotypes that “mimic” the ligand and, as a consequence, bind to and neutralize the receptor and/or its ligands. Such neutralizing anti-idiotypes or Fab fragments of such anti-idiotypes can be used in therapeutic regimens to neutralize a ligand and/or its receptor. Methods employing the use of polynucleotides comprising a nucleotide sequence encoding an engineered antibody or a fragment thereof are provided herein.

In one embodiment, the nucleotide sequence encoding an antibody that specifically binds an antigen is obtained and used to generate the engineered antibodies disclosed herein. The nucleotide sequence can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+RNA, isolated from any tissue or cells expressing the antibody, such as hybridoma cells selected to express an antibody) by PCR amplification using synthetic primers that hybridize to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999)), to generate antibodies having a different amino acid sequence by, for example, introducing deletions, and/or insertions into desired regions of the antibodies.

In a specific embodiment, one or more of the CDRs is inserted within framework regions using routine recombinant DNA techniques. The framework regions may be naturally occurring or consensus framework regions, including, but not limited to, human framework regions (see, e.g., Chothia et al., 1998, J. Mol. Biol. 278:457-479 for a listing of human framework regions). It is contemplated that the polynucleotide generated by the combination of the framework regions and CDRs encodes an antibody that specifically binds to an Antigen. In one embodiment, as discussed supra, one or more amino acid substitutions may be made within the framework regions, and, in certain embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

The hinge of antibodies identified from such screening methods can be modified as described supra to generate an antibody incorporating a modified hinge, such as any of those disclosed above. It is further contemplated that the engineered antibodies disclosed herein are useful for the prevention, management and treatment of a disease, disorder, infection, including but not limited to inflammatory diseases, autoimmune diseases, bone metabolism related disorders, angiogenic related disorders, infection, and cancer. Such antibodies can be used in the methods and compositions disclosed herein.

### B. Recombinant Expression

Recombinant expression of any of the engineered antibodies disclosed herein as well as derivatives, analogs or fragments thereof, (e.g., an antibody or fusion protein of the invention), requires construction of an expression vector containing a polynucleotide that encodes the engineered antibody. Once a polynucleotide encoding an engineered antibody has been obtained, the vector for the production of the engineered antibody can be produced by recombinant DNA technology using techniques well known in the art. Thus, methods for preparing a protein by expressing a polynucleotide containing engineered antibody-encoding nucleotide sequence are described herein. Methods that are well known to those skilled in the art can be used to construct expression vectors containing engineered antibody coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce an engineered antibody (such as any of those disclosed herein). In specific embodiments for the expression of engineered antibodies comprising double-chained antibodies, vectors encoding both the heavy and light chains may be co-expressed in the host cell for expression of the entire immunoglobulin molecule.

### C. Administration of Pharmaceutical Compositions

Also provided herein are methods and pharmaceutical compositions comprising any of the engineered antibodies disclosed herein (such as any antibody comprising the modified hinge regions disclosed herein). Further provided herein are methods of treatment, prophylaxis, and amelioration of one or more symptoms associated with a disease, disorder or infection by administering to a subject an effective amount of at least one engineered antibody disclosed herein, or a pharmaceutical composition comprising at least one engineered antibody disclosed herein. In a one aspect, the engineered antibody is substantially purified (i.e., substantially free from substances that limit its effect or produce undesired side-effects). In a specific embodiment, the subject is an animal, such as a mammal including non-primates (e.g., cows, pigs, chickens or other fowl, horses, cats, dogs, rats etc.) and primates (e.g., monkey such as, a cynomolgous monkey and a human). In a specific embodiment, the subject is a human. In yet another specific embodiment, the antibody of the invention is from the same species as the subject.

The route of administration of the composition depends on the condition to be treated. For example, intravenous injection may be preferred for treatment of a systemic disorder such as a lymphatic cancer or a tumor which has metastasized. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Depending on the condition, the composition can be administered orally, parenterally, intranasally, vaginally, rectally, lingually, sublingually, buccally, intrabuccally and/or transdermally to the patient.

Accordingly, compositions designed for oral, lingual, sublingual, buccal and intrabuccal administration can be made without undue experimentation by means well known in the art, for example, with an inert diluent or with an edible carrier. The composition may be enclosed in gelatin capsules or compressed into tablets. For the purpose of oral therapeutic administration, the pharmaceutical compositions of the present invention may be incorporated with excipients and used in the form of tablets, pellets, troches, capsules, elixirs, suspensions, syrups, wafers, chewing gums, and the like. In further embodiments, the composition can be incorporated into an animal feed.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Non-limiting examples of excipients include starch and lactose. Some examples of disintegrating agents include alginic acid, cornstarch, and the like. Examples of lubricants include magnesium stearate and potassium stearate. An example of a glidant is colloidal silicon dioxide. Some non-limiting examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The pharmaceutical compositions disclosed herein can be administered parenterally, such as, for example, by intravenous, intramuscular, intrathecal and/or subcutaneous injection. Parenteral administration can be accomplished by incorporating the compositions disclosed herein into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic. Rectal administration includes administering the composition into the rectum and/or large intestine. This can be accomplished using suppositories and/or enemas. Suppository formulations can be made by methods known in the art. Transdermal administration includes percutaneous absorption of the composition through the skin. Transdermal formulations include patches, ointments, creams, gels, salves, and the like. The engineered antibody-containing compositions disclosed herein can be administered nasally to a patient. As used herein, nasally administering or nasal administration includes administering the compositions to the mucous membranes of the nasal passage and/or nasal cavity of the patient.

The engineered antibody-containing compositions disclosed herein can be used in accordance with the methods of the invention for preventing, treating, or ameliorating one or more symptoms associated with a disease, disorder, or infection. It is contemplated that the pharmaceutical compositions of the invention are sterile and in suitable form for administration to a subject.

In one embodiment, the engineered antibody-containing compositions disclosed herein are pyrogen-free formulations which are substantially free of endotoxins and/or related pyrogenic substances. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26(1):223 (2000)). When therapeutic proteins are administered in amounts of several hundred or thousand milligrams per kilogram body weight, as can be the case with monoclonal antibodies, it is advantageous to remove even trace amounts of endotoxin. In a specific embodiment, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

Additionally provided herein are methods for preventing, treating, or ameliorating one or more symptoms associated with a disease, disorder, or infection, said method comprising: (a) administering to a subject in need thereof a dose of a prophylactically or therapeutically effective amount of a composition comprising one or more of the engineered antibodies disclosed herein and (b) administering one or more subsequent doses of said engineered antibodies, to maintain a plasma concentration of the engineered antibodies at a desirable level (e.g., about 0.1 to about 100 μg/ml), which continuously binds to an antigen. In a specific embodiment, the plasma concentration of the engineered antibodies is maintained at 10 ng/ml, 15 g/ml, 20 μg/ml, 25 μg/ml, 30 μg/ml, 35 μg/ml, 40 μg/ml. 45 μg/ml or 50 μg/ml. In a specific embodiment, said effective amount of engineered antibodies to be administered is between at least 1 mg/kg and 8 mg/kg per dose. In another specific embodiment, said effective amount of engineered antibodies to be administered is between at least 4 mg/kg and 8 mg/kg per dose. In yet another specific embodiment, said effective amount of engineered antibodies to be administered is between 50 mg and 250 mg per dose. In still another specific embodiment, said effective amount engineered antibodies to be administered is between 100 mg and 200 mg per dose.

Also provided herein are protocols for preventing, treating, or ameliorating one or more symptoms associated with a disease, disorder, or infection which an any of the engineered antibodies disclosed herein is used in combination with a therapy (e.g., prophylactic or therapeutic agent). The engineered antibodies disclosed herein can potentiate and synergize with, enhance the effectiveness of, improve the tolerance of, and/or reduce the side effects caused by, other cancer therapies, including current standard and experimental chemotherapies. The combination therapies of the invention have additive potency, an additive therapeutic effect or a synergistic effect. The combination therapies of the invention enable lower dosages of the therapy (e.g., prophylactic or therapeutic agents) utilized in conjunction with the engineered antibodies disclosed herein for preventing, treating, or ameliorating one or more symptoms associated with a disease, disorder, or infection and/or less frequent administration of such prophylactic or therapeutic agents to a subject with a disease disorder, or infection to improve the quality of life of said subject and/or to achieve a prophylactic or therapeutic effect. Further, the combination therapies of the invention reduce or avoid unwanted or adverse side effects associated with the administration of current single agent therapies and/or existing combination therapies, which in turn improves patient compliance with the treatment protocol. Numerous molecules which can be utilized in combination with the engineered antibodies disclosed herein are well known in the art. See for example, PCT publications WO 02/070007; WO 03/075957 and U.S. Patent Publication 2005/064514.

### IV. Kits

Further provided herein are kits comprising one or more of the engineered antibodies disclosed herein with altered (such as, improved) stability and/or decreased potential for proteolytic cleavage that specifically bind to an antigen conjugated or fused to a detectable agent, therapeutic agent or drug, in one or more containers, for use in monitoring, diagnosis, preventing, treating, or ameliorating one or more symptoms associated with a disease, disorder, or infection.

The invention can be further understood by reference to the following examples, which are provided by way of illustration and are not meant to be limiting.

### EXAMPLES

### Example 1: Generation and Evaluation of Site Evaluation Libraries

### A. Plasmids and Site Evaluation Construction for Anti-RSV HC Heavy Chain

Sequences for the heavy chain of monoclonal antibodies against respiratory syncytial virus (palivizumab or Synagis) were codon optimized and synthesized by GeneArt GmH (Germany). To prevent from potential degradation by Kex2 furin-like protease during expression in a fungal cell, a lysine at position 216 in the heavy chain was mutated to threonine (K216T). Initially synthetic sequences of the c2G4 and anti-RSV HC were cloned individually behind a catalytical core of the Trichoderma reesei native cellobiohydrolase I (CBH1) together with its linker region (1-479 aa). To release mature antibody chains from the carrier partner a Kex2 cleavage site was introduced between the linker and HC.

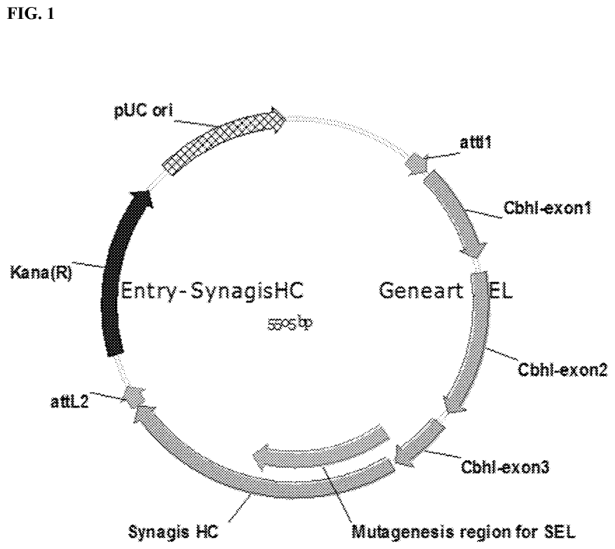

A fusion construct of cbhI-HC_Synagis was then amplified by PCR with gene specific primers extended with the attB1 and attB2 sites to allow for the Gateway® BP recombination cloning into pDonor221 vector (Invitrogen, USA). Plasmid pEntry-SynagisHC_Geneart_SEL, as shown in FIG. 1, was used by the vendor BaseClear (Netherlands) as a template for construction of site evaluation (SEL) library at positions 466-725 aa (counting from the CbhI Met). An average number of mutant variants per aa position was around 17. Mutated sequences were further cloned via the Gateway® LR recombination technique into pTTTpyr2-IScel destination vector resulting in the final expression plasmids pTTTpyr2-IScel-SynagisHC_Geneart_SEL (FIG. 1).

This expression vector contains the T. reesei cbhI promoter and terminator regions allowing for a strong inducible expression of a gene of interest and the T. reesei pyr2 selective marker conferring growth of transformants on minimal medium without supplementation with uridine. The plasmid is maintained autonomously in fungal cells due to T. reesei derived telomere regions. Plasmids were propagated in commercially available Escherichia coli TOP10 cells (Invitrogen, US), purified, sequence verified, arrayed individually in 96 well MTPs and used for fungal transformation as described below.

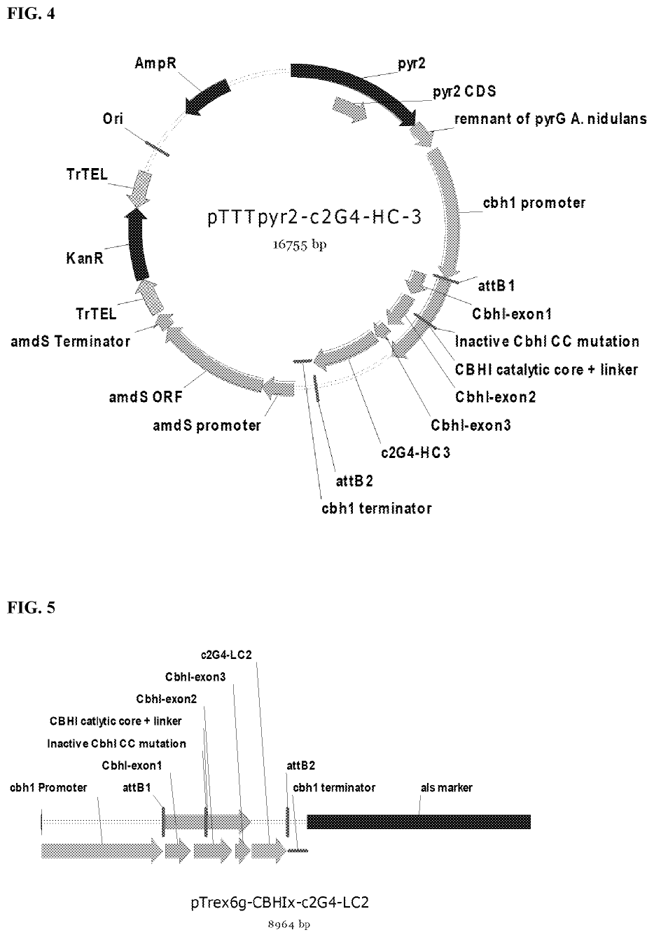

pEntry-Synagis_LC_Geneart plasmid was constructed via the Gateway® BP recombination cloning and recombined further with pTrex6g destination vector in a similar way as described above resulting in the expression vector pTrex6g-Synagis_LC. This vector served as a template to generate a PCR fragment expressing the light chain (FIG. 4).

The anti-RSV antibody was made as described in International Patent Application No. PCT/US2020/021685, the disclosure of which is incorporated by reference herein in its entirety. An anti-herpes simplex virus antibody (HSV08), an anti-HIV antibody (VRC01), and an anti-HER2/neu antibody (Trastuzumab) were made in a similar manner.

### B. Fungal Host Strain Construction and Transformation

The expression cassette consists of a CBH1 promoter, CBH1 core, antibody HC and LC connected by CBH1 linker and kex2 sequence for processing of CBH1, CBH1 terminator, and the alS marker conferring resistance to chlorimuron ethyl to a fungal cell. The alS marker was used for making screening strains so that the pyr2 marker was available for the SEL variants. The full expression cassette was amplified by PCR. The PCR product was cleaned up and concentrated to 500-1000 ng/μL. The expression cassette was randomly integrated into the host T. reesei genome at multiple copies, as described below.

The host T. reesei strain used for transformation was deleted for major cellulases and xylanases. The strain was transformed using a standard PEG-protoplast transformation method. Transformation mixtures containing approximately 10 μg of DNA and 5×10⁶ protoplasts in a total volume of 250 μl were treated with 2 mL of 25% PEG solution, diluted with 2 volumes of 1.2M sorbitol/10 mM Tris, pH7.5/10 mM CaCl2 solution, and mixed with 26 mL of 2% low melting agarose containing 1M sorbitol, 1 g/L uridine, 75 mg/L chlorimuron ethyl in minimal medium and distributed over four 10 cm petri plates pre-poured containing 1.5% agarose, 1M sorbitol in minimal media. After sufficient growth transformants from each plate were observed, individual colonies were picked onto fresh 10 cm petri plates containing 1.5% agar, 1 g/L uridine, 75 mg/L chlorimuron ethyl, 4 per plate to allow room for assessing stability. The stable colony phenotype is concentric circular growth with smooth edges. Once stable transformants were observed and well sporulated, spores were harvested and used for inoculation of liquid cultures to evaluate expression level of LC by Western blot analysis using a light chain specific antibody peroxidase conjugate from Sigma-Aldrich (USA). One transformant #LC6 with a high expression level of LC served as a screening host for further expression of the heavy chain SEL library.

All high throughput transformations with Synagis HC variants were performed robotically in a 24 well MTP format using Biomek robots (Beckman Coulter, USA). Plasmids with variants were received from the vendor in a 96 well format arrayed according to a predetermined layout. Transformation mixtures containing approximately 1 μg of DNA and 5×10⁶ protoplasts of the screening host #LC6 in a total volume of 50 μl were treated with 200 μl of 25% PEG solution, diluted with 1 volumes of 1.2M sorbitol/10 mM Tris, pH7.5/10 mM CaCl2 solution, rearranged robotically into 24 well MTPs and poured in 1 ml of 3% low melting agarose containing 1M sorbitol in minimal medium. After sufficient growth transformants from each well were pooled together and plated on fresh 24 well agar plates with minimal medium. Once sporulated, spores were harvested and used for inoculation of liquid cultures.

Fungal host strain construction and transformation for the anti-RSV antibody was made as described in International Patent Application No. PCT/US2020/021685, the disclosure of which is incorporated by reference herein in its entirety. Host strain construction and transformation were performed for an anti-herpes simplex virus antibody (HSV08), an anti-HIV antibody (VRC01), and an anti-HER2/neu antibody (Trastuzumab) in a similar manner.

### C. Fungal Fermentations in Slow Release 24 Well MTPs

To generate sufficiently high antibody titers 10⁵-10⁶ T. reesei spores were inoculated in customer made 24 well MTPs composed of the Sylgard 170 elastomer (from Dow Corning, USA) premixed with lactose which was slowly released in the medium during fermentation to ensure continuous production. Cultures were grown in 1.25 ml of medium containing: 16 g/L glucose, 9 g/L casamino acids, 10 g/L (NH4)2SO4, 4.5 g/L KH₂PO4, 1 g/L MgSO4*7H2O, 1 g/L CaCl2*2H2O, 33 g/L PIPPS buffer [pH 5.5], 0.25% T. reesei trace elements (100%: 175 g/L citric acid (anhydrous), 200 g/L FeSO4*7H2O, 16 g/L ZnSO4*7H2O, 3.2 g/L CuSO4*5H2O, 1.4 g/L MnSO4*H2O, 0.8 g/L H3BO3).

Plates were incubated in Infors shaker with a 50 mm throw at 200 rpm and 28 C with 80% humidity. After 5-6 days of growth cultures were reformatted back to 96 well deep well MTPs and filtered using 96-well microtiter filter plates (0.2 μm hydrophilic PVDF membrane, Corning, Tewksbury MA). The plates were frozen in Axygen half-deep well plates (P-DW-11-C).

D. miniSEL Screen

Plasmids and library construction: Sequences for humanized version of Zmap c2G4 monoclonal antibodies against Ebola virus were codon optimized and synthesized by GeneArt GmH (Germany). To prevent from potential degradation by Kex2 furin-like protease during expression in a fungal cell, all KR sites were removed from both heavy (HC) and light (LC) chains. Initially synthetic sequences of the c2G4 HC and LC were cloned individually behind a catalytically inactive core of the Trichoderma reesei native cellobiohydrolase I together with its linker region (1-479 aa). To release mature antibody chains from the carrier partner a Kex2 cleavage site SVAVEKR was introduced between the linker and either HC or LC.

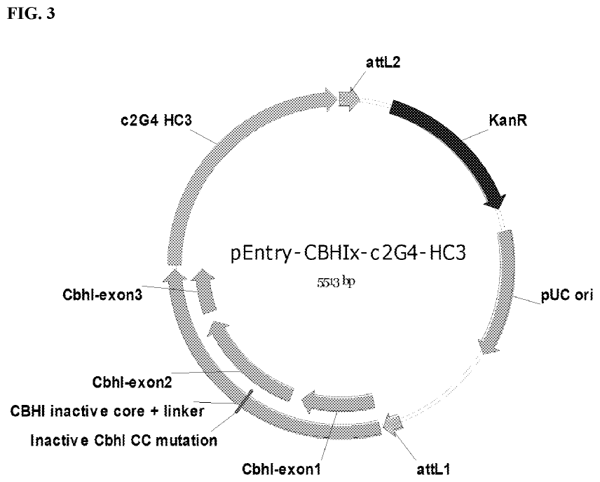

Fusion constructs of cbhI-c2G4 HC3 and cbhI-c2G4_LC2 were then amplified by PCR with gene specific primers extended with the attB1 and attB2 sites to allow for the Gateway® BP recombination cloning into pDonor221 vector (Invitrogen, USA). Plasmid pEntry-CbhIx-c2G4_HC3, as shown in FIG. 3, was used by the vendor BaseClear (Netherlands) as a template for construction of site evaluation (SEL) library at positions 217-236 aa (counting on mature HC). A number of mutant variants per each aa position varied between 13 and 19 with an average number above 16. Mutant variants were further cloned via the Gateway® LR recombination technique into pTTTpyr2 destination vector resulting in the final expression plasmids pTTTpyr2-CbhI-c2G4_HC3 (FIG. 3).

This expression vector contains the T. reesei cbhI promoter and terminator regions allowing for a strong inducible expression of a gene of interest, the Aspergillus nidulans amdS and T. reesei pyr2 selective markers conferring growth of transformants on minimal medium with acetamide in the absence of uridine. The plasmids are maintained autonomously in fungal cells due to T. reesei derived telomere regions. Usage of replicative plasmids results in increased frequencies of transformation and circumvents problems of locus-dependent expression observed with integrative fungal transformation. Plasmids were propagated in commercially available Escherichia coli TOP10 cells (Invitrogen, US), purified, sequence verified, arrayed individually in 96 well MTPs and used for fungal transformation as described below.

pEntry-CbhIx-c2G4_LC2 plasmid was recombined with pTrex6g destination vector in a similar way as described above resulting in the expression vector pTrex6g-CbhIx-c2G4_LC2. This vector served as a template to generate a PCR fragment expressing c2G4_LC2 driven by the cbhI promoter and linked to the alS marker conferring resistance to chlorimuron ethyl to a fungal cell (FIG. 4).

Fungal strains and transformation: Prior to screening of the c2G4_HC3 heavy chain SEL library in T. reesei, an intermediate strain #C1 expressing sufficient levels of the c2G4_LC2 light chain was constructed. With this purpose, a light chain expression fragment was randomly integrated in a T. reesei strain deleted for major cellulases and xylanases using a standard PEG-protoplast transformation method. Transformants resistant to 50-80 mg/L of chlorimuron ethyl due to the presence of the alS marker connected to c2G4_LC2 were screened for LC expression via a combination of ELISA and Western blot assays using a light chain specific antibody peroxidase conjugate from Sigma-Aldrich (USA). One transformant, labelled #C1, with a strong signal of LC on a Western blot served as a host for further expression of the c2G4_HC3 SEL library.

All high throughput transformations with the c2G4_HC3 variants were performed robotically in a 24 well MTP format using Biomek robots (Beckman Coulter, USA). Plasmids with variants were received from the vendor in a 96 well format arrayed according to a predetermined layout. Transformation mixtures containing approximately 1 μg of DNA and 5×10⁶ protoplasts of the screening strain #C1 in a total volume of 50 μl were treated with 200 μl of 25% PEG solution, diluted with 1 volumes of 1.2M sorbitol/10 mM Tris, pH7.5/10 mM CaCl2 solution, rearranged robotically into 24 well MTPs and poured in 1 ml of 3% low melting agarose containing 1M sorbitol in minimal medium. After sufficient growth transformants from each well were pooled together and plated on fresh 24 well agar plates with minimal medium containing 10 mM acetamide as a sole nitrogen source. Once sporulated, spores were harvested and used for inoculation of liquid cultures.

Fungal fermentations in slow release 24 well MTPs: To generate sufficiently high antibody titers 10⁵-10⁶ T. reesei spores were inoculated in customer made 24 well MTPs composed of the Sylgard 170 elastomer (from Dow Corning, USA) premixed with lactose which was slowly released in the medium during fermentation to ensure continuous production. Cultures were grown in 1.25 ml of medium containing: 16 g/L glucose, 9 g/L casamino acids, 10 g/L (NH4)2SO4, 4.5 g/L KH₂PO4, 1 g/L MgSO4*7H2O, 1 g/L CaCl2*2H2O, 33 g/L PIPPS buffer [pH 5.5], 0.25% T. reesei trace elements (100%: 175 g/L citric acid (anhydrous), 200 g/L FeSO4*7H2O, 16 g/L ZnSO4*7H2O, 3.2 g/L CuSO4*5H2O, 1.4 g/L MnSO4*H2O, 0.8 g/L H3BO3).

Plates were incubated in Infors shaker with a 50 mm throw at 200 rpm and 28 C with 80% humidity. After 5-6 days of growth cultures were reformatted back to 96 well deep well MTPs and filtered using 96-well microtiter filter plates (0.2 μm hydrophilic PVDF membrane, Corning, Tewksbury MA). Clarified samples were analyzed for the expression of antibodies.

### Example 2: Evaluation of Antibody Hinge Variants for Resistance to Cleavage

Clipping Assay: This method measures the amount of antibody proteolysis following expression and purification from a host cell. The concentration of the purified antibody variants produced in Example 1 was determined by either BCA assay (C2G4 samples) for total protein or by a FRET assay (antiRSV, HSV08, VRC01, and Trastuzumab samples). The BCA assay was performed as described by the manufacturer (Thermo Fisher Scientific 23225). The C2G4 antibodies were then normalized to 60 ppm, and the FRET quantitated antibodies were normalized to 120 ppm. Some of the antibodies were not normalized due to their purified concentrations being lower than the concentration the other were normalized to. The dilution buffer used for the normalization was a Glycine-Tris buffer that was at the same pH and concentration as the solution of the antibodies after purification.

An empty Cellulighter host strain was run in Dasgip under the same conditions that hinge clipping is observed. The supernatant was isolated and concentrated roughly 10-fold.

For the C2G4 antibodies, 100 μL of 60 ppm protein normalized sample was added to a Corning 3605 plate. For the FRET quantitated antibodies, the antibody samples were diluted 4-fold with Mili-Q water before adding them to a Corning 3605 plate. 10 μL of the concentrated cellulighter broth was then added to each well to start the hinge clipping reaction. The plate was sealed with BioRad Microseal B and placed at 25° C. in an Eppendorf thermomixer. The plates incubated for 100 minutes with shaking. The reaction was quenched by mixing 50 μL of reaction mixture and 50 μL protease inhibitor cocktail in a 3605 plate. The protease inhibitor cocktail was Halt™ Protease Inhibitor Cocktail (Fisher 78430) with added EDTA at a final concentration that was 10× the recommended dilution. These stressed samples were then stored on ice until they were processed by western analysis. Aliquots of the antibody samples at the same dilution as in the reaction but without any concentrated broth were mixed in the same ratio with the protease inhibitor cocktail as the stressed samples to generate a T₀ timepoint (unstressed).

The unstressed and stressed samples were analyzed for hinge clipping via western blot. The samples were run on Invitrogen E-PAGE 48-well 8% gels, transferred to nitrocellulose membrane by Invitrogen iBlot 2, and were probed using the Invitrogen iBind Flex. For a more accurate comparison, the unstressed and stressed samples for a given variant were run next to each other on the same row of the 48-well gel. Anti-Human Fc-HRP (Sigma A0170) was used to probe the samples, in conjunction with SuperSignal™ West (Thermo 34076). The blots were visualized and quantitated by the BioRad ChemiDoc MP and its software. The percentage of clipped calculated by dividing the volume of the bottom band (Fc-fragment from the clipped HC) by the sum of the three fragments (CBH1-HC, mature-HC, Fc-fragment). The reported delta in hinge clipping is the difference in percent hinge clipping before and after the samples incubated with the concentrated Cellulighter broth.

Purification: Plates were moved from the freezer to the cold room to allow the samples to gradually thaw overnight at 4° C. Before purification, grown WT samples were removed from the plates and these samples were pooled. One mL per well of pooled WT, pooled low binding control, pooled high binding control, and pooled vector only (vector expressing CBH1 in same strain) samples were added to designated wells. The library plates were grown in duplicate and these controls were added to both plates. The plates gently shook for 2 minutes to homogenize the fluid in the wells followed by centrifugation for 1 minute to pellet any precipitate.

The robot handled four library plates at a time. The robot added 50 μL of 1 M KPi pH 7 to pH up the supernatant to improve the antibody binding to the Protein A resin. The robot then transferred the crude material (max 880 μL per well) from the four plates to 2 mL filter plates (Pall 8275) filled previously with 220 μL of Protein A resin in PBS. These filter plates then shook for 5 minutes on a shaker. The plates were then filtered by centrifugation at 1000 g for 2 minutes, and the flow through was collected in the empty harvest plate that the samples were transferred from. This material was stored until after quantitation. The filter plates were returned to the robot deck and the duplicate growth plates were added to the same filter plates. These plates were incubated and centrifuged as before. The resin was then washed with 880 μL of PBS buffer. The plates shook for 1 minute and then centrifuged at 1000 g for 2 minutes. The flow through was discarded, and the plates were returned to the robot for the second PBS washing. After the second washing, the plates were moved to a robot running the elution program.

The elution program handled four plates at a time. It added 11 μL of neutralization buffer (1 M Tris pH 9) to a clean half-deep well plate that the samples would be eluted into. The program then added 440 μL of elution buffer (100 mM glycine pH 2.7) to the filter plates. The plates then shook for 1 minute at setting 7 and then were filtered by centrifugation (1000 g for 2 minutes) into the freshly prepped recovery plates. After centrifugation, the sample plates shook for 1 minute to ensure proper mixing of the neutralization buffer.

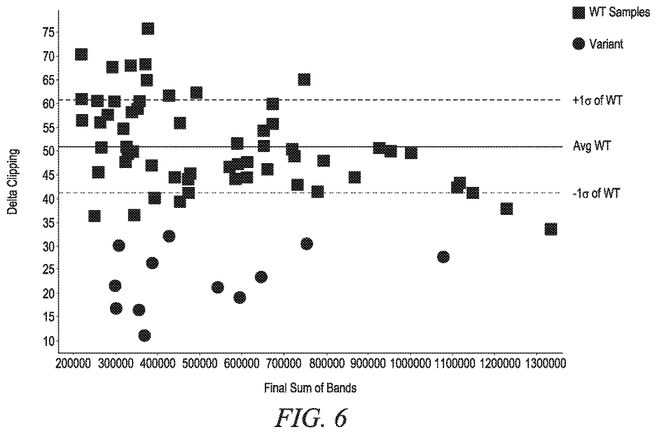

Results: For the C2G4 antibody, as shown in FIG. 6, several hinge sequence variants were identified that resulted in significantly less antibody clipping compared to mature antibodies that were not modified in the hinge region. A complete listing of these variants as well as the assayed decrease in hinge clipping is shown in Table 3.

TABLE 3
Results of clipping assay for C2G4 antibody

| Mutations* | Delta Clipping |
|---|---|
| K216T | 23 |
| K216V | 30 |
| T217S | 28 |
| S222C | 16 |
| S222D | 17 |
| S222E | 21 |
| T226N | 26 |
| T226P | 19 |
| H227P | 11 |
| A234R | 30 |
| Average WT | 51 |

*The sequence positions listed for the C2G4 clipping data in TABLE 3 were changed to reflect their corresponding position with respect to SEQ ID NO: 1. This change was based on the sequence alignment shown in FIG. 9 for the two hinge regions.

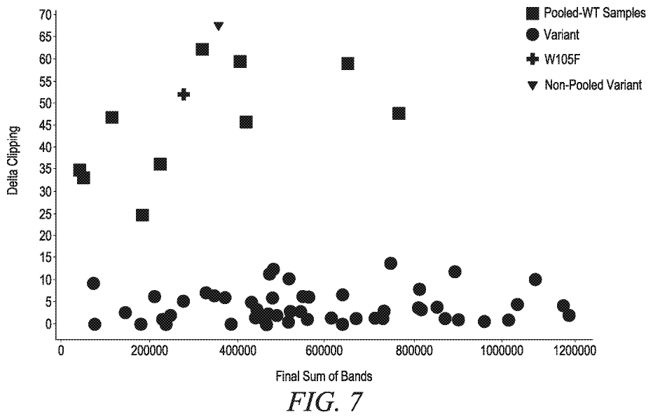

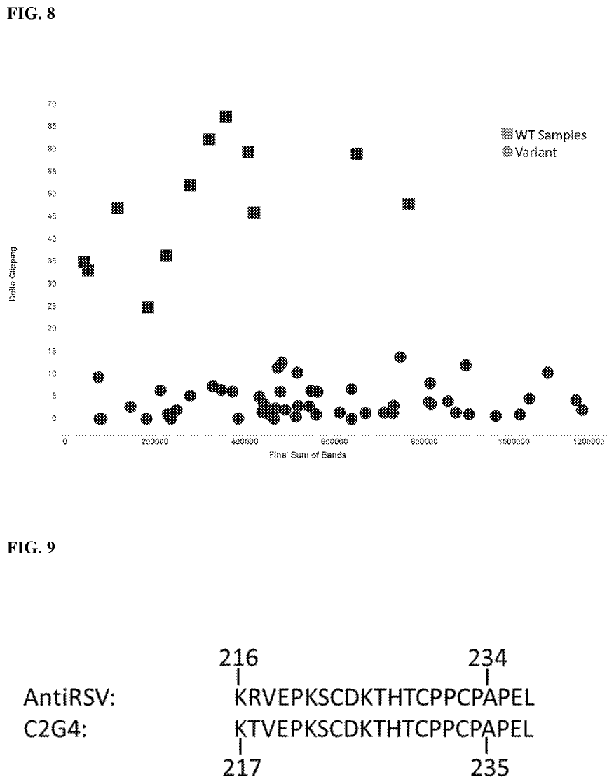

When the substitutions found to reduce clipping in the C2G4 antibody shown in Table 3 were introduced into the hinge of the anti-RSV antibody, as shown in FIG. 7 and FIG. 8, these mutations also resulted in significantly less antibody clipping compared to mature antibodies that were not modified in the hinge region, demonstrating that these hinge substitutions reduce clipping across multiple antibodies. A complete listing of these C2G4 variants as well as the assayed decrease in hinge clipping is shown in Table 4.

TABLE 4
Results of clipping assay for RSV antibody

| | Delta |
|---|---|
| Mutations | Clipping |
| R217S-T226N | 14 |
| R217S-S222C | 13 |
| T216V-R217S-T226N | 12 |
| S222C-H227P | 11 |
| H227P | 10 |
| R217S-S222E-H227P | 10 |
| T216V-R217S-S222C-T226P | 9.2 |
| T226P-H227P | 8.0 |
| R217S-S222C-T226P | 7.3 |
| T216V-S222D-T226P | 6.6 |
| T216V-S222C-T226P-H227P | 6.4 |
| T226N | 6.3 |
| T216V-S222E-T226N | 6.2 |
| T216V-S222C-H227P | 6.1 |
| R217S-S222C-T226N | 6.1 |
| S222E | 6.0 |
| S222C-T226P-H227P | 5.2 |
| T216V-S222C-T226N-H227P | 4.9 |
| R217S-S222D-T226P-H227P | 4.5 |
| T216V-H227P | 4.1 |
| S222E-T226P-H227P | 3.9 |
| R217S-S222D-T226N-H227P | 3.7 |
| R217S-S222E-T226P-H227P | 3.3 |
| T216V-S222C | 3.3 |
| T226P | 2.9 |
| R217S-S222D | 2.9 |
| S222E-T226P | 2.8 |
| R217S-S222C-T226P-H227P | 2.7 |
| T216V-T226P-H227P | 2.3 |
| T216V-S222D-H227P | 2.2 |
| T226N-H227P | 2.1 |
| S222D-T226N-H227P | 1.9 |
| R217S-S222E-T226N | 1.9 |
| R217S-T226N-H227P | 1.5 |
| R217S-S222E-T226N-H227P | 1.4 |
| T216V-R217S-H227P | 1.4 |
| T216V-S222E-T226P-H227P | 1.3 |
| T216V-S222C-T226N | 1.3 |
| R217S-T226P-H227P | 1.2 |
| S222C | 1.2 |
| S222D | 1.0 |
| T216V-T226P | 1.0 |
| S222E-T226N-H227P | 1.0 |
| S222E-H227P | 0.9 |
| R217S-S222E-T226P | 0.6 |
| R217S-T226P | 0.5 |
| T216V-S222D- | 0.1 |
| R217S-S222C-H227P | 0 |
| T216V-S222E-T226N-H227P | 0 |
| S222C-T226N-H227P | 0 |
| T216V-R217S-S222C-T226N- | 0 |
| H227P | |
| R217S-S222D-T226P | 0 |
| S222D-T226N | 0 |
| Average Pooled WT | 44 |

When select substitutions found to reduce clipping in the C2G4 antibody were introduced into the hinge of an anti-herpes simplex virus antibody (HSV08), an anti-HIV antibody (VRC01), and an anti-HER2/neu antibody (Trastuzumab), these mutations also resulted in significantly less antibody clipping compared to mature antibodies that were not modified in the hinge region, further demonstrating that these hinge substitutions reduce clipping across multiple antibodies (Table 5).

TABLE 5
Results of clipping assay for additional antibodies

| Mutation | HSV08 | VRC01 | Herceptin |
|---|---|---|---|
| 222C | 36.5 | 0 | 15.3 |
| 222D | 7.8 | 0 | 5.3 |
| 222E | 35.1 | 0 | 10.5 |
| 226N | 31.5 | 10.8 | 14.9 |
| 226P | 24.4 | 0 | 0.3 |
| 227P | 84.1 | 10.6 | 2.4 |
| WT | 80.0 | 80.2 | 82.0 |

### Example 3: Evaluation of CHO-Expressed Antibody Hinge Variants for Resistance to Cleavage

CHO Produced antiRSV Variants: Chinese hamster ovary (CHO) cell expressed variants were obtained from Bionova Scientific (Fremont, CA). The variants were delivered purified and in PBS buffer. The concentration was measured by the FRET assay using Synagis, and the variants were diluted to 120 ppm with the same Tris-Gly as before.

FRET Quantitation Assay and Normalization: Protein A (Thermo Fisher Scientific 77674) was labeled with Alexa Fluor 546 NHS ester (Thermo Fisher Scientific A20102). Protein L (Thermo Fisher Scientific 77680) was labeled with Alexa Fluor 488 NHS ester (Thermo Fisher Scientific A20100). The labeled Protein A and Protein L were diluted with 107 mM KPi pH 7 and at a ratio that produced a FRET signal for our standard curve with the proper dynamic range. The standard curve was commercial Synagis from Abb Vie. In a Corning 3605 plate, 40 μL of the Protein A and L solution was mixed with 10 μL of the purified antibody sample. The FRET signal on the plate was read (ex: 485 nm em: 590 nm cutoff: 590 nm), and the concentration of the unknowns was determined from the Synagis standard curve. The samples were run in duplicate.

After analyzing the data, the plates were normalized to 120 ppm. The dilution buffer was Tris-Gly buffer that was at the same pH and concentration is in the purified samples. For the wells that were less than 120 ppm, they were not diluted and were used as is. Results are shown in Table 6. As shown, CHO expressed anti-RSV variants exhibited significantly less clipping compared to the wild type antibody hinge.

TABLE 6
Data for CHO Expressed antiRSV

| Mutations | Delta Clipping |
|---|---|
| S222E | 7.6 |
| T226N | 11.4 |
| H227P | 0.5 |
| WT | 76.5 |

SEQUENCES

QVTLRESGPALVKPTQTLTLTCTFSGFSLSTSGMSVGWIRQPPGKALEWLADIWWDDKK
DYNPSLKSRLTISKDTSKNQVVLKVTNMDPADTATYYCARSMITNWYFDVWGAGTTV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDK/TRVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK (SEQ ID NO: 1)
Nucleotide sequence of Synagis HC:
caggtcaccctccgcgagagcggccctgctctcgtcaagcccacgcagaccctcacgctgacctgcaccttcagcggcttcagcctcagc
accagcggcatgagcgtcggctggatccgccagcctcctggcaaggccctcgagtggctcgccgacatctggtgggacgacaagaagg
actacaaccccagcctcaagagccgcctcaccatcagcaaggacaccagcaagaaccaggtcgtcctgaaggtcaccaacatggacccc
gccgacaccgccacctactactgcgcccgcagcatgatcaccaactggtacttcgacgtctggggcgctggcacgaccgtcaccgtcagc
agcgcctccacgaagggccccagcgtctttccgctcgctcccagcagcaagagcacctccggcggcacggctgccctcggctgcctggt
caaggactacttccccgagcctgtcacggtcagctggaactctggcgccctgaccagcggcgtccacacgttccccgccgtcctccagag
cagcggcctctactccctcagcagcgtcgtcacggtccccagcagctccctcggcacccagacctacatctgcaacgtcaaccacaagcc
tagcaacaccaaggtcgacacccgcgtcgagcccaagagctgcgacaagacccacacgtgccctccgtgccctgctcctgagctgcttg
gcggcccttcggtctttctgttccctccgaagcctaaggacaccctcatgatctcgcgcacgcccgaggtcacgtgcgtcgtcgtcgacgtc
agccacgaggaccctgaggtcaagtttaactggtatgtcgacggcgtcgaggtccacaacgctaagacgaagccccgcgaggagcagta
caacagcacctaccgcgtcgtcagcgtcctcaccgtcctgcaccaggactggctcaacggcaaggagtacaagtgcaaggtcagcaaca
aggccctgcctgctcctatcgagaagaccatctccaaggccaagggccagcctcgcgagccccaggtctacaccctgcctccgagccga
gaggagatgacgaagaaccaggtgagcctgacctgcctcgtcaagggcttctaccccagcgacattgccgtcgagtgggagagcaacg
gccagcctgagaacaactacaagaccacgcctcctgtcctcgactccgacggctcgttcttcctgtacagcaagctcaccgtcgacaagtc
ccgctggcagcagggcaacgtctttagctgcagcgtcatgcacgaggccctccacaaccactacacccagaagtccctctcgctcagccc
cggcaagtaa (SEQ ID NO: 2)
Amino Acid sequence of Synagis HC:
QVTLRESGPALVKPTQTLTLTCTFSGFSLSTSGMSVGWIRQPPGKALEWLADIWWDDKK
DYNPSLKSRLTISKDTSKNQVVLKVTNMDPADTATYYCARSMITNWYFDVWGAGTTV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDTRVEPKSCDKTHTCPPCPAPE
LLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK (SEQ ID NO: 3)
Nucleotide sequence of Synagis LC:
gacatccagatgacgcagagccccagcacgctcagcgccagcgtcggcgaccgcgtcaccatcacgtgcaagtgccagctctccgtcg
gctacatgcactggtatcagcagaagcccggcaaggcccctaagctcctcatctacgacaccagcaagctcgccagcggcgtccccagc
cgattctccggctctggctccggcaccgagttcaccctcaccatcagctcgctgcagcccgacgacttcgccacctactactgcttccaggg
ctcgggctaccccttcaccttcggcggcggcacgaagctcgagatcaagcgcaccgtcgccgctcctagcgtctttatcttcccgcctagcg
acgagcagctcaagagcggcaccgcctccgtcgtctgcctgctcaacaacttctacccgcgcgaggccaaggtccagtggaaggtcgac
aacgccctccagagcggcaactcccaggagagcgtcaccgagcaggactccaaggacagcacctacagcctcagcagcaccctcacg
ctctccaaggccgactacgagaagcacaaggtctacgcctgcgaggtcacccaccagggcctgagcagccccgtcaccaagagcttcaa
ccgcggcgagtgctaa (SEQ ID NO: 4)
Amino Acid sequence of Synagis LC:
DIQMTQSPSTLSASVGDRVTITCKCQLSVGYMHWYQQKPGKAPKLLIYDTSKLASGVPS
RFSGSGSGTEFTLTISSLQPDDFATYYCFQGSGYPFTFGGGTKLEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC (SEQ ID NO: 5)
Nucleotide sequence of the c2G4_HC3:
gaggtccagctccaggagagcggcggcggcctcatgcagcccggcggcagcatgaagctctcctgcgtcgccagcggcttcaccttca
gcaactactggatgaactgggtccgccagagccccgagaagggcctcgagtgggtcgccgagatccgcctcaagagcaacaactacgc
cacccactacgccgagagcgtcaagggccgcttcaccatcagccgcgacgacagcaagaactccgtctacctccagatgaacaccctcc
gcgccgaggacaccggcatctactactgcacgcgcggtaacggcaactaccgcgccatggactactggggccagggcaccagcgtcac
ggtctccagcgccagcaccaagggcccaagcgtctttcccctcgcccccagcagcaagagcaccagcggcggcaccgccgccctcgg
ctgcctcgtcaaggactacttccccgagcccgtcactgtcagctggaacagcggcgctctcaccagcggcgtccacaccttccccgccgtc
ctccagagcagcggcctctacagcctcagcagcgtcgtcaccgtccccagcagcagcctcggcacccagacctacatctgcaacgtcaa
ccacaagcccagcaacaccaaggtcgacaagaccgtcgagcccaagagctgcgacaagacccacacctgccccccctgccccgcccc
cgagctgctcggcggcccctccgtctttctcttcccccccaagcccaaggacaccctcatgatcagccgcacccccgaggtcacctgcgtc
gtcgtcgatgtcagccacgaggaccccgaggtcaagttcaactggtacgtcgacggcgtcgaggtccacaacgccaagaccaagccccg
cgaggagcagtacaacagcacctaccgcgtcgtcagcgtcctgaccgtcctccaccaggactggctcaacggcaaggagtacaagtgca
aggtctccaacaaggccctccccgcccccatcgaaaagaccatcagcaaggccaagggccagccccgcgagccccaggtctacaccct
cccccccagccgcgaggagatgaccaagaaccaggtctccctcacctgcctggtcaagggcttctaccccagcgacatcgccgtcgagt
gggagagcaacggccagcccgagaacaactacaagaccaccccccccgtcctcgacagcgacggcagcttcttcctctacagcaagctc
accgtcgacaagagccgctggcagcagggcaacgtattagctgcagcgtcatgcacgaggccctccacaaccactacacccagaagag
cctcagcctcagccccggcaagtaa (SEQ ID NO: 6)
Amino Acid sequence of the c2G4_LC2:
EVQLQESGGGLMQPGGSMKLSCVASGFTFSNYWMNWVRQSPEKGLEWVAEIRLKSNN
YATHYAESVKGRFTISRDDSKNSVYLQMNTLRAEDTGIYYCTRGNGNYRAMDYWGQG
TSVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKTVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (SEQ ID NO: 7)
Nucleotide sequence of the c2G4_LC2:
Gacatccagatgacccagagccccgccagcctcagcgtctctgtcggcgagaccgtctccatcacctgccgcgccagcgagaacatcta
cagcagcctcgcctggtatcagcagaagcagggcaagagcccccagctcctcgtctacagcgccaccatcctcgccgacggcgtcccca
gccgcttcagcggcagcggcagcggcacccagtacagcctcaagatcaacagcctccagagcgaggacttcggcacctactactgcca
gcacttctggggcaccccctacaccttcggcggcggcaccaagctggagatcacccgcaccgtcgcggcgccaagcgtattatcttccc
ccccagcgacgagcagctcaagagcggcaccgccagcgtcgtctgcctcctcaacaacttctacccccgcgaggccaaggtccagtgga
aggtcgacaacgccctccagagcggcaacagccaggagagcgtcaccgagcaggacagcaaggactccacctacagcctcagcagca
ccctcaccctctccaaggccgactacgagaagcacaaggctacgcctgcgaggtcacccaccagggcctcagctcccccgtcaccaag
agcttcaaccgcggcgagtgctaa (SEQ ID NO: 8)
Amino Acid sequence of the c2G4_HC3 LC:
DIQMTQSPASLSVSVGETVSITCRASENIYSSLAWYQQKQGKSPQLLVYSATILADGVPS
RFSGSGSGTQYSLKINSLQSEDFGTYYCQHFWGTPYTFGGGTKLEITRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 9)

1. A monoclonal IgG1 antibody heavy chain polypeptide comprising a hinge region that comprises one or more amino acid modification(s) that reduces proteolysis of the polypeptide.

2. The polypeptide of claim 1, further comprising an IgG1 antibody light chain polypeptide.

3. The polypeptide of claim 1, wherein the modification(s) comprise a modification at one or more of amino acid positions 216, 217, 222, 226, and/or 234, wherein the amino acid positions are numbered according to the numbering in SEQ ID NO:1.

4. The polypeptide of claim 3, wherein the modifications comprise one or more of 216T or V; 217T or S; 222C, D, or E; 226N or P; and/or 234R.

5. The polypeptide of claim 1, wherein the modification further comprises a modification at position 227.

6. The polypeptide of claim 5, wherein the modification comprises 227P.

7. The polypeptide of claim 3, wherein the modifications comprise a modification at position 216 and one or more modifications at amino acid positions 222, 226, 227, and/or 234.

8. The polypeptide of claim 7, wherein the modifications comprise 216T and one or more of 222C, D, or E; 226N or P; 227P; and/or 234R.

9. The polypeptide of claim 3, wherein the modifications comprise a modification at position 217 and one or more modifications at amino acid positions 222, 226, 227, and/or 234.

10. The polypeptide of claim 7, wherein the modifications comprise 217T and one or more of 222C, D, or E; 226N or P; 227P; and/or 234R.

11. The polypeptide of claim 1, wherein the modification is a combinatorial modification selected from the group consisting of: (a) R217S-T226N; (b) R217S-S222C; (c) T216V-R217S-T226N; (d) S222C-H227P; (e) R217S-S222E-H227P; (f) T216V-R217S-S222C-T226P; (g) T226P-H227P; (h) R217S-S222C-T226P; (i) T216V-S222D-T226P; (j) T216V-S222C-T226P-H227P; (k) T216V-S222E-T226N; (1) T216V-S222C-H227P; (m) R217S-S222C-T226N; (n) S222C-T226P-H227P; (o) T216V-S222C-T226N-H227P; (p) R217S-S222D-T226P-H227P; (q) T216V-H227P; (r) S222E-T226P-H227P; (s) R217S-S222D-T226N-H227P; (t) R217S-S222E-T226P-H227P; (u) T216V-S222C; (v) R217S-S222D; (w) S222E-T226P; (x) R217S-S222C-T226P-H227P; (y) T216V-T226P-H227P; (z) T216V-S222D-H227P; (aa) T226N-H227P; (bb) S222D-T226N-H227P; (cc) R217S-S222E-T226N; (dd) R217S-T226N-H227P; (ee) R217S-S222E-T226N-H227P; (ff) T216V-R217S-H227P; (gg) T216V-S222E-T226P-H227P; (hh) T216V-S222C-T226N; (ii) R217S-T226P-H227P; (jj) T216V-T226P; (kk) S222E-T226N-H227P; (11) S222E-H227P; (mm) R217S-S222E-T226P; (nn) R217S-T226P; (00) T216V-S222D; (pp) R217S-S222C-H227P; (qq) T216V-S222E-T226N-H227P; (rr) S222C-T226N-H227P; (ss) T216V-R217S-S222C-T226N-H227P; (tt) R217S-S222D-T226P; and (uu) S222D-T226N (vv).

12. The polypeptide of claim 1, wherein said polypeptide exhibits at least about 50% less proteolysis compared to a monoclonal IgG1 antibody heavy chain polypeptide that does not comprise said one or more amino acid modifications.

13. The polypeptide of claim 1, wherein said polypeptide exhibits no detectable proteolysis.

14. The polypeptide of claim 1, further comprising a polypeptide encoding a signal sequence.

15. The polypeptide of claim 1, further comprising a polypeptide encoding a carrier protein.

16. The polypeptide of claim 14, wherein the polypeptide encoding a carrier protein is adjacent to the polypeptide encoding a signal sequence.

17. The polypeptide of claim 14, wherein the carrier protein comprises CBH1 or a fragment thereof.

18. The polypeptide of claim 1, wherein the antibody is an anti-Respiratory Syncytial Virus (RSV) antibody, an anti-ebola virus antibody, an anti-aggregated β-amyloid (AB) antibody, an anti-human immunodeficiency virus (HIV) antibody, an anti-herpes simplex virus (HSV) antibody, an anti-sperm antibody (such as an anti-human contraceptive antigen (HCA) antibody), or an anti-HER2/neu antibody.

19. The polypeptide of claim 1, wherein the polypeptide exhibits increased stability compared to a monoclonal IgG1 antibody heavy chain polypeptide that does not comprise said one or more amino acid modifications.

20. A nucleic acid encoding the fusion polypeptide of any one of claim 1.

21-45. (canceled)
